(19) United States
(12) Patent Application Publication
(10) Pub. No.: US 2010/0249048 A1
(43) Pub. Date: Sep. 30, 2010

(54) BETA GLYCOLIPIDS FOR THE TREATMENT OF CALCIFICATION RELATED DEGENERATIVE DISORDERS

(76) Inventors: Yaron Ilan, Jerusalem (IL); Haim Lotan, Ramat-Gan (IL); Mony Shuvy, Zur Hadassah (IL); Gadi Lalazar, Mevaseret Zion (IL)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/746,430

(22) PCT Filed: Dec. 7, 2008
(86) PCT No.: PCT/IL08/01587

§ 371 Date: Jun. 4, 2010

(30) Foreign Application Priority Data
Dec. 6, 2007 (IL) 187959

(51) Int. Cl. A61K 31/7028; A61P 13/12; C12N 5/02

(52) U.S. Cl. 514/25; 514/42; 435/375

(57) ABSTRACT

The present invention relates to a composition comprising a combination of at least one natural or synthetic β-glycolipid, a mixture of at least two β-glycolipids, a substance which increases the intracellular, extracellular or serum level of a naturally occurring β-glycolipid, or any combination or mixture thereof and at least one phosphate inhibitor or any compound that alters the phosphate binding or transporting into or out of any cell or any membrane or any combination thereof. The combined composition of the invention may be particularly used for treating calcification related degenerative disorders, specifically, vascular and valvular disorders. The invention further provides kits methods and uses thereof for treatment of calcification related disorders.

### FIELD OF THE INVENTION

The invention relates to the use of β-glycolipids, natural or synthetic analogs, derivatives thereof and any mixture or combination thereof, particularly with phosphate inhibitors, for the treatment and prevention of calcification related degenerative disorders, specifically, vascular and valvular disorders.

### BACKGROUND OF THE INVENTION

All publications mentioned throughout this application are fully incorporated herein by reference, including all references cited therein.

Pathogenesis of Aortic valve Calcification (AVC)

Valvular heart disease is the name given to any dysfunction or abnormality of one or more of the heart's four valve including the mitral valve, aortic valve, tricuspid valve and pulmonic valve. According to the American Heart Association 2006 Heart and Stroke Statistical Update [Thom T. et al. Circulation 113:e85-151 (2006)], valvular heart disease is responsible for nearly 20,000 deaths each year in the United States and is considered a contributing factor in about 42,000 deaths. The majority of these cases involve disorders of the aortic valve (63 percent).

There are a number of types of valvular heart disease, including: valvular stenosis, valvular regurgitation, atresia and mitral valve prolapse. Valvular stenosis is a condition in which there is narrowing, stiffening, thickening, fusion or blockage of one or more valves of the heart. The defective narrowed (stenosed) valve can interfere with the smooth passage of blood. Of the four types of valvular stenosis, which correspond to the four types of heart valves, aortic stenosis (AS) is the most common type of valvular heart diseases in general. Aortic stenosis can be caused by congenital heart disease or by the buildup of calcium. This buildup causes thickening and stiffening of the valve cusps and leads to decreased mobility and fusion of the valve's flaps.

Aortic valve disease is the most common disease in the elderly, characterized by aortic valve calcification (AVC) and regional valve thickening. Ultimately, about 10% of patients with the slowly progressive disorder of calcific aortic stenosis (CAS) will develop severe calcification and significant aortic stenosis (AS). Aortic sclerosis has two important clinical implications. First, it may be an antecedent to clinically significant aortic valve obstruction, and second, it acts as a marker of increased risk of cardiovascular events. Current treatments include valve replacement or balloon valvuoloplasty.

In the past, AVC was considered to be a degenerative disease, caused by tissue necrosis and calcium precipitation. Recent studies show an active process of calcification, involving differentiation of valve myofibroblasts into osteoblasts, resulting in increased expression of bone proteins: osteopontin, osteocalcin and runx2 [Rajamannan, N. et al. Circulation 107:2181-2184 (2003)]. It was suggested that osteoblast differentiation is mediated by inflammatory process, since activated T cells and macrophages were identified in aortic valve lesions together with several cytokines (TGF-β, TNFα and others) [Kaden J. J., Cardiovasc. Pathol. 14(2):80-87 (2005)].

Active calcification is prominent early in the disease process and is a major factor in the leaflet stiffness of severe stenosis. Histopathologic studies of aortic sclerosis show focal subendothelial plaque-like lesions on the aortic side of the leaflet that extend to the adjacent fibrosa layer. These lesions share similarities to atherosclerosis, with accumulation of “atherogenic” lipoproteins, including LDL and lipoprotein(a), evidence of LDL oxidation an inflammatory cell infiltrate, and microscopic calcification[10]. Inflammatory cells are the predominant cell type in early aortic valve lesions, with T lymphocytes and macrophages identified. Monocytes infiltrate the endothelial layer via adhesion molecules and differentiate into macrophages. Activated T lymphocytes within the subendothelium and fibrosa release cytokines, such as TGF-β1, increasing local production of matrix metalloproteins, all of which contribute to extracellular matrix formation, remodeling, and local calcification. Recent data suggest that the final calcification is an active process, involving valve myofibroblasts that differentiate into osteoblasts. Osteoblast differentiation is mediated by an inflammatory process.

The molecular basis of calcification was studied using various tissues. Variety of cell surface G-proteins coupled receptors were considered to activate calcification process in bone tissue. The activation of these receptors, results in upregulation of RANK/RANK ligand (RANK/RANKL), which are part of the TNF family pathway (NFκB cell machinery) [Hofbauer, L. C., and A. E. Heufelder. Journal of molecular medicine (Berlin, Germany) 79:243-253 (2001); Schoppet, M., Arteriosclerosis, Thrombosis, and Vascular Biology 22:549-553 (2002)]. RANK (Receptor Activator of Nuclear Factor κ B) is a transmembrane protein expressed on the surface of osteoclasts (cells involved in bone resorption), which together with its ligand was shown to play an important role in osteoclast activation, osteoclast differentiation and calcification [Hamdy, N. A. Curr. Opin. Investig. Drugs 8:299-303 (2007)]. RANK ligand (Receptor Activator for Nuclear Factor κ B ligand) activates osteoclasts as well as serve as a major activator of runx-2, which is the most important transcription factor for osteoblast differentiations [Wu, X. et al. The Journal of Clinical Investigation 112:924-934 (2003)]. Overproduction of RANKL is implicated in a variety of degenerative bone diseases, such as rheumatoid arthritis and osteomyelitis. RANKL also has a function in the immune system, where it is expressed by T helper cells and is thought to be the major activator of the NFκB pathway involved. Recently several studies showed high expression of RANKL in human calcified aortic valves [Kaden, J. et al. Journal of Molecular and Cellular Cardiology 36:57-66 (2004)]. In cultured human aortic valve myofibroblasts, RANKL was shown to promote both matrix calcification as well as the transition towards an osteogenic phenotype, suggesting that the expression of the RANK/RANKL have a regulatory role not only in osteocalstogenesis but also in calcification [Shetty, R. et al. Heart (British Cardiac Society) 92:1303-13088, (2006); Kaden (2004) ibid.]. Influencing RANKL expression has therefore become a target of the inventor's research.

OPG ligand (OPGL) regulates lymph node organogenesis, lymphocyte development, and interactions between T cells and dendritic cells in the immune system. OPGL expression in T cells is induced by antigen-receptor engagement, which suggests that activated T cells may influence bone metabolism through OPGL and RANK. Activated T cells can directly trigger osteoclastogenesis through OPGL, and RANK provides critical signals necessary for lymph node organogenesis and osteoclast differentiation.

Parathyroid Hormone

PTH is a peptide hormone which is the most important regulator of calcium and phosphate metabolism. It is essential for bone formation, osteoblast activity, and for vitamin D activation [Cheung J. et. al. J. Endocrinol 177:423-433 (2003)]. Activation of PTH receptor induces osteocalcin, runx-2, and RANKL, therefore stimulating osteocalstogenesis and calcification [Partridge N. C. et al. Ann. N. Y. Acad. Sci. 1068:187-193 (2006)]. In addition to osteoblast and osteoclast activation, PTH has several immune modulatory and pro inflammatory roles as well as many effects on the heart (to alter heart rate, coronary blood flow, peak pressure and rate of rise of left ventricular pressure)[Silverberg S. J. Clin. Endocrinol Metab. 86(10):3513-351412 (2000)]. Its effect on lymphocyte function was shown in both components of the immune system, though the exact effect on T cell population is controversial. Studies conducted on dialysis patients showed significant correlation between PTH level, valve calcification and inflammatory markers. Both increased and decreased secretions of PTH are recognized as causes of serious disease. Excessive secretion of PTH is seen in two forms: primary hyperparathyroidism and secondary hyperthyroidism. Both forms are associated with abnormalities in vascular function and decreased vascular compliance which in turn is linked to increased cardiovascular risk.

Renal Failure and AVC

Evidence for the relationship between renal dysfunction and adverse cardiovascular events was first recognized in the dialysis population in whom the incidence of cardiovascular death is strikingly high. There is growing evidence that relatively minor renal abnormalities may be associated with increased risk of cardiovascular events. One of the principal pathophysiological mechanisms involved in this association has been proposed to be endothelial dysfunction which is recognized as one of the initial mechanisms that lead to atherosclerosis.

Accelerated calcifying atherosclerosis and valvular heart disease occur with high frequency in chronic kidney disease patients [Schiffrin, E. L et al. Circulation 116:85-97 (2007)]; Impaired endothelial function may explain the high prevalence of coronary ischemia (inadequate blood flow) and cardiovascular events in these patients. Progressive deterioration of renal function in chronic kidney disease patients may also lead to dyslipidemia. This lipid disorder involves the elevation of total cholesterol, LDL cholesterol and triglycerides levels each have been correlated with accelerated arterial occlusive diseases. Dyslipidemia can stimulate oxidative stress and inflammation, which in turn may contribute to endothelial dysfunction and progression of arthrosclerosis.

Renal failure is a significant risk factor for AVC and aortic stenosis, and 40% of the patients who suffer from end stage renal disease have AVC. Renal failure causes an elevated phosphate level, decreased calcium level and secondary hyperparathyroidism. As a result of these metabolic changes these patients have diffused calcification and formation of hydroxyapetite crystals in several tissues including the aortic valve.

The Role of the Immune System in the Pathogenesis of Vascular Disorders and Atherosclerosis

Atherosclerosis, also known as “hardening of the arteries”, can occur in any artery in the body. The disease is a chief contributor to cardiovascular disease, the leading cause of death among men and women in the United States. According to the American Heart Association 2007 Heart and Stroke Statistical Update [Rosamond W. et al. Circulation. 115:e69-171(2007)], atherosclerosis accounts for nearly 75 percent of all deaths from cardiovascular disease. All individuals over the age of 65 are especially prone to developing advanced atherosclerosis.

Atherosclerosis is a chronic disease that causes various cardiovascular complications. Although the initiation and progression of atherosclerosis largely depend on genetic factors and life styles, the cellular and molecular mechanisms are still not clear. Nonetheless, in recent years, scientists have made great strides forward in understanding the atherosclerotic process as recent studies have revealed that cellular and humoral immunity play crucial roles in atherogenic lesion formation, including macrophages, CD4+ and CD8+ T cells and dendritic cells as well as auto antigens such as heat shock protein (HSP 60/65) and oxidized LDL. Various modifications of the immune system have been proposed as therapeutic strategies, with the potential of inhibiting atherosclerosis progression. The aim of these: modifications, induced by specific vaccination, is to switch CD4+ T cells from Th1 toward a Th2 anti-inflammatory cytokine secretion, and to induce protective antibodies [Ohashi, R., et al. Med. Sci. Monit. 10(11):RA255-60 (2004)]. PTH plays a role in atherosclerosis as increased risk of cardiovascular disease has been reported in patients with primary hyperparathyroidism (PHPT). Several studies demonstrated a direct effect of PTH on vascular wall thickening and plaque progression and increases proinflammatory cytokines secretion.

The Role of the Immune System in the Pathogenesis of Valvular Disorders

The early lesion of “degenerative” aortic stenosis is an active inflammatory process, calcific and atherosclerotic processes occur at the vascular level, so that vascular and valvular degeneration share a number of similarities, such as: inflammation; lipid infiltration; dystrophic calcification; ossification, platelet deposition; and endothelial dysfunction [Mohler, E. R. 3^{rd} et al. Arterioscler Thromb. Asc. Biol. 17:547-552 (1997); Hunt J. L. et al. Stroke 33:1214-1219 (2002)]. The presence of osteopontin in calcified human aortic valves suggests that osteopontin is a regulatory protein in pathologic calcification. PTH has an immunomudulatory effect, and seems to affect lymphocyte function and especially cellular immunity. PTH acts as a growth factor and inflammatory mediator in rheumatic diseases, and was recently shown to activate T cells (particularly Th2) and the NFκB system [Mohler E. R. 3^{rd} et al. Circulation. 103:1522-1528 (2001); Young N., et al. J. Immunol. 175(12):8287-8295 (2005).

Most Western countries face high and increasing rates of vascular diseases. Each year, heart diseases kill more Americans than cancer. Disease of the heart alone caused 30% of all deaths, with other diseases of the cardiovascular system causing substantial further death and disability. Up until the year 2005, vascular diseases were the number1 cause of death and disability in the United States and most European countries. A large histological study (PDAY) showed vascular injury accumulates from adolescence, making primary prevention efforts necessary from childhood [McGill H. C. Jr. et. al. Arterioscler Thromb. Vasc. Biol. 20:1998-2004 (2000)]. By the time that vascular, problems are detected, the underlining cause (atherosclerosis) is usually quite advanced, having Progressed for decades. There is therefore increased emphasis on the prevention of vascular disorders.

The Role of the Immune System in Pathological Calcification

Injury to the endothelial lining of an organelle may lead to pathological calcification. During inflammation resulting from such injury, growth factors and inflammatory mediators released by platelets induce leukocyte invasion; the proliferation of resident cells and, the up-regulation of various cytokines, including osteopontin, by smooth muscle cells and macrophages. Although tissue integrity is restored, occasionally the process becomes pathological, resulting in excessive cell proliferation and the deposition of an extracellular matrix molecule that in time becomes calcified.

Recent studies have shown that osteopontin has both pro- and anti-inflammatory actions and a defined role in regulating inflammatory cell accumulation and function at sites of inflammation and repair. These studies indicate that osteopontin posses a critical role in the recruitment of macrophages and production of certain cytokines during cell-mediated immunity as the protein interacts with integrins and CD44 to enhance Th1 and inhibit Th2 cytokine expression. The expression of osteopontin was found to be enhanced in a variety of inflammatory processes (such as atherosclerosis) which also involve calcification. Moreover, NKT cells, which play a critical role in several inflammatory diseases such as atherosclerosis, are an important cellular source of osteopontin [Diao H. et. al. Immunity. 21:539-550 (2004)].

Other studies indicate osteopontin exerts anti-inflammatory effects, influences tissue repair at sites of inflammatory response and may function as a negative regulator of calcification, by virtue of its abilities to inhibit the production of pro-inflammatory NO and prostaglandin E2, and to strongly bind calcium phosphate crystals in mineralized tissues, inhibiting crystal growth [Denhardt D. T. et al. J. Clin. Invest. 107(9):1055-1061 (2001)].

It is therefore possible that osteopontin play a protective role where necessary and expressed in atherosclerotic plaques only in response to local conditions that might tend to favor calcium mineralization [Doherty T. M. et al. Proc. Natl. Acad. USA 100(20): 11201-11206 (2003)].

Current therapies to normalize serum mineral levels or to decrease, inhibit or prevent calcification of vascular and valvular tissues are of limited efficacy. Therefore, there exist a need for an effective method of inhibiting and preventing vascular and valvular calcification.

β-glucosylceramide (β-GC) and β-lactosylceramide (β-LC) are metabolic intermediates in the metabolic pathways of complex glycoglycosphingolipids. IGL is the combination of these compounds and was recently suggested to exert an immune modulatory effect in various inflammatory disorders. The inventors have recently showed, as disclosed by WO2007/060652, that a novel combination of β-glucosylceramide with β-lactosyl-ceramide in a 1:1 ratio (IGL), is effective in immune-modulation and alleviation of immune-related disorders.

Surprisingly, the inventors now show that J3-glycolipid, and particularly, a mixture of both β-lactosyl-ceramide and β-glucosylceramide affect intracellular mechanisms involving the RANK pathways, and therefore may have an effect on calcium metabolism. The possible involvement of β-glycolipids in calcium metabolism may therefore be applicable for treating disorders related to calcification processes, such as vascular and valvular degenerative disorders. The inventors further showed that the calcification process is dynamic and reversible and that targeting components involved in phosphate metabolism, for example, the sodium-phosphate co transporter PIT1, alleviates AVC.

The present invention therefore provides a comprehensive approach of targeting both, the immunomodulatory and the calcification aspects. Thus, it is an object of the invention to provide combined compositions and kits integrating the immunomodulatory effect of β-glycolipids, particularly, the IGL, with PTH or phosphate inhibitors.

As shown by the invention such compositions are particularly useful in the treatment of calcification related disorders, specifically, vascular and valvular degeneration disorders.

Another object of the invention is to provide methods using β-glycolipids and mixtures thereof, optionally with phosphate inhibitors, in the treatment of subjects suffering from vascular or valvular degenerative disorders, particularly atherosclerosis-related disorders as well as from valvular heart diseases, particularly calcific aortic valve stenosis which dramatically increase the risk of mortality

These and other objects of the invention will become clearer as the description proceeds

### SUMMARY OF THE INVENTION

According to a first aspect, the invention relates to a composition comprising a combination of at least one natural or synthetic β-glycolipid, a mixture of at least two (3-glycolipids, a substance which increases the intracellular, extracellular or serum level of a naturally occurring β-glycolipid, or any combination or mixture thereof and at least one phosphate inhibitor or any compound that alters the phosphate binding or transporting into or out of any cell or any membrane or any combination thereof. It should be noted that the composition of the invention may optionally further comprises at least one pharmaceutically acceptable carrier, diluent, excipient and/or additive.

According to one embodiment, the composition of invention may be particularly used for the treatment of a calcification related degenerative disorder.

Another aspect of the invention relates to a kit for achieving a therapeutic effect in a subject in need thereof, specifically for treating calcification related degenerative disorder. According to this aspect, the kit of the invention comprises: (a) at least one natural or synthetic β-glycolipid, a mixture of at least two β-glycolipids, a substance which increases the intracellular, extracellular or serum level of a naturally occurring β-glycolipid; or any combination or mixture thereof, or a pharmaceutically acceptable derivative thereof and a pharmaceutically acceptable carrier or diluent, optionally, in a first unit dosage form; (b) at least one phosphate inhibitor any compound that alters the phosphate binding or transporting into or out of any cell or any membrane or any combination thereof and a pharmaceutically acceptable carrier or diluent, optionally, in a second unit dosage form; and (c) container means for containing said first and second dosage forms.

A further aspect of the invention relates to a method for the treatment or prevention of a calcification related degenerative disorder in a subject in need thereof. According to one embodiment, the method of the invention comprises the step of administering to said subject a therapeutically effective amount of at least one of: (a) at least one of a natural or synthetic β-glycolipid, a mixture of at least two β-glycolipids, a substance which increases the intracellular, extracellular or serum level of a naturally occurring β-glycolipid; (b) at least one phosphate inhibitor or any compound that alters the phosphate binding or transporting into or out of any cell or any membrane or any combination thereof; and (c) a composition or kit comprising (a) or (b) or of any combinations thereof.

In yet a further aspect, the invention relates to the use of a therapeutically effective amount of a least one of: (a) any one of a β-glycolipid, a mixture of at least two β-glycolipids, and a substance which increases the intracellular, extracellular or serum level of a naturally occurring β-glycolipid or of any-combinations and mixtures thereof; (b) at least one phosphate inhibitor or any compound that alters the phosphate binding or transporting into or out of any cell or any membrane or any combination thereof; and (c) any combination or mixture of (a) or (b), in the preparation of a pharmaceutical composition for the treatment or prevention of a calcification related degenerative disorder in a subject in need thereof.

The invention further provides a method for protection of cells of the endothelial lining from a vascular or valvular degenerative process using the combined compositions, kits of the invention or any one of β-glycolipid, phosphate inhibitors or any combinations thereof.

Still further, the invention provides a method for decreasing RANKL expression in a subject in need thereof, and thereby blocking TNF family pathway, using the combined compositions, kits of the invention or any one of β-glycolipid, phosphate inhibitors or any combinations thereof.

These and other aspects of the invention will become apparent by the hand of the following examples.

### BRIEF DESCRIPTION OF THE FIGURES

FIG. 1: The expression levels of RANKL

The effect of IGL on RANKL expression in aortic valves was assessed by western blot for RANKL expression performed in aortic valve tissues obtained from the three groups of rats. A significant decrease in expression of RANKL protein level following IGL administration is shown. Abbreviations: Exp. (experimental), gr. (group).

FIG. 2: The effect of IGL on aortic valve NKT cells.

Aortic valves NKT cells were determined in experimental groups using FACS analysis. AVC was associated with a significant increase in valvular NKT cells (group A) while IGL administration in Group B, altered the NKT distribution pattern which was similar to that of the controls (group C). Abbreviations: Aor. (Aortic), Va. (Valve's), Lyra. (Lymphocyte), Exp. (Experimental), gr. (group). Percentages were calculated by FACS analysis performed on lymphocytes isolated from the valve.

FIG. 3A-3B: The effect of IGL on aortic valve CD8 cells

FIG. 3A. Histogram presenting the effect of IGL on aortic valve CD8 cells: CD8 cells were isolated from aortic valves and sorted by FACS. AVC was associated with a significant increase in intra-valvular CD8 cells (group A) while IGL administration (group B) altered the lymphocyte distribution pattern in a way similar to that noted in control animals (group C).

FIG. 3B. A representative FACS analysis of NKT and CD8 lymphocytes isolated from aortic valves.

Abbreviations: Aor. (Aortic), Va. (Valve's), Lym. (Lymphocyte), Exp. (Experimental), gr. (group), Ad. Phos. (adenine phosphate diet group), cont. (control). Percentages were calculated by FACS analysis.

FIG. 4: MSCT analysis of AVC rats treated with IGL

MSCT was used to assess the effect of IGL treatment on valve calcification. Valve calcification was quantified using the Agatston Score. AVC was identified in all animals from groups A and B that received a high-adenine (0.75%), high-phosphorus diet (1.5%). No calcification was found in the controls (group C, not shown). Treatment with IGL led to a significant decrease in AVC in group B treated animals compared with group A. Abbreviations: Exp. (experimental), gr. (group), ca (calcium), score (score).

FIG. 5A-5B:. Chest MSCT

A representative chest MSCT of a rat from group A showing the heart and bony structures (white arrows in A and B). Calcified tissues, including the ribs and vertebrae, are stained in pink in both images. Calcium aggregates are demonstrated in the diet group rat aortic valve annulus (yellow arrow in A) and is not seen in the IGL-treated group in group B (yellow arrow, B).

FIG. 6A-6C: IGL reduces apoptosis

Aortic valves were stained for cleaved caspase 3. A representative sections of aortic valves of a rat from control group C, and from IGL-treated group in group B show decrease staining compared with calcified valves in group A.

FIG. 7: Phosphate treatment induces calcification in myofibroblasts.

Von Kossa stain for calcium aggregates (brown) demonstrates calcification only myofibroblasts treated with phosphate. Abbreviations: cont (control), P (phosphate).

FIG. 8 Expression of PIT 1 in aortic valve myofibroblasts

Immunohistochemical staining of myofibroblasts using anti-PIT1 antibodies.

FIG. 9 Foscarnet inhibits Phosphate the phosphate induced calcification in myofibroblasts

Von kossa staining of myofibroblasts treated either with phosphate or with phosphate and foscarnet as compared to untreated control myofibroblasts.

FIG. 10 Forscarnet decreases AVC in treated rats

MSCT was used to assess the effect of Forscarnet treatment on valve calcification. Valve calcification was quantified using the Agatston Score. AVC was identified in all animals received a high-adenine (0.75%), high-phosphorus diet (1.5%) (Ade. Pho.). Treatment with Forscarnet led to a significant decrease in AVC. Abbreviations: ca (calcium), score (score), Ad. (adenine), ph. (phosphate).

### DETAILED DESCRIPTION OF THE INVENTION

As shown by the following examples, administration of glycosphingolipids to animals with AVC, and specifically of a mixture of β-lactosyl-ceramide and β-glucosyl-ceramide (designated by the invention as IGL), resulted in a clear alteration of the CD8 and NKT lymphocytes distribution in the valve and decreased RANKL expression. These alterations were associated with a significant alleviation of the aortic stenosis.

Native AVC is an inflammatory process that includes accumulation of activated T cells and macrophages in aortic valve lesions. Administration of β-glycosphingolipids to animals with AVC and the consequent decreased RANKL expression prevented the intra-valve accumulation of CD8 and NKT lymphocytes. These immune changes were associated with a significant alleviation of AVC.

Induction of valve calcification by a high-adenine, high-phosphorus diet in the present study was associated with a significant increase in RANKL expression in aortic valves. Administration of IGL led to a significant reduction in RANKL expression. These changes reflect activation of NFκB family members in the valve tissue during calcification as well as prevention of this process by IGL. RANKL and OPG (osteoprotegerin) are differentially expressed in calcific AS. In cultured human aortic valve myofibroblasts, RANKL promotes matrix calcification and induces expression of osteoblast-associated genes, indicating a transition towards an osteogenic phenotype, supporting a potential role for the RANKL-OPG pathway in regulating AVC. The expression pattern of the RANKL/RANK/OPG system also suggests that it may have a regulatory role not only in osteoclastogenesis but also in the calcification of aortic valves.

Studies on the pathogenesis, of calcific aortic valve disease support an active inflammatory disease process with lipoprotein deposition and chronic and osteoblast futures in valve tissue. AVC was used to be considered as an irreversible process. However, in recent years, efforts have been made to slow its progression. A role for immune modulation in the alleviation of the calcification process in AVC has not been previously investigated Immune modulatory approaches aimed at T-cell activation or trafficking, however, have decreased leaflet cellular infiltration and prevented allograft valve structural failure.

Valve lesions in degenerative CAS (calcific aortic stenosis) are infiltrated by T lymphocytes. The present invention indicates that CD8 accumulation in the valve may be important in the development of AVC. A significant increase in CD8 lymphocytes was noted in valves from animals with the disease as compared with control valves. Suppression of RANKL expression by IGL inhibited the intra-valvular CD8 cell accumulation. Without being bound by any theory, the inventors hypothesize that this inhibition may be associated with a preventive effect on CD8 accumulation in the valve. A similar effect of glycoshpingolipids on CD8 lymphocytes has been previously described by part of the inventors in other models WO 2007/060652]. The RANK-dependent IGL inhibition of CD8 lymphocytes, shown by the present invention, was associated with alleviation of the aortic calcification as quantified by MSCT.

Analysis of the T lymphocytes infiltrating the calcified valves exhibited features of a polyclonal nonselective response to inflammation or contained expanded clones, suggesting a more specific immune process. The CD43+, CD3+, and CD8+ infiltrates have been suggested to be associated with valvular structural damage in an animal model of allograft. A study in human calcified valves showed activated CD8+ T cell accumulation close to endothelial cells that produced interferon-γ and may be involved in AVC. Recent data suggest that clonally expanded alphabeta T cells are implicated in mediating a component of the valvular injury responsible for CAS. The TCR β-chain CDR3-length distribution analysis using PCR primers specific for twenty three Vbeta families revealed considerable oligoclonal T cell expansion among infiltrated T lymphocytes from degenerative CAS valves. These findings suggest that clonally expanded alphabeta T cells are implicated in mediating a component of the valvular injury responsible for CAS.

The present invention further shows that the high-adenine, high-phosphorus diet was associated with a significant increase in NKT regulatory cells in the valve, as compared with control valves. Suppression of RANKL expression by IGL inhibited the intra-valvular NKT cell accumulation and was associated with alleviation of the degree of valve calcification. A similar inhibitory effect of β-glycosphingolipids in NKT-mediated immune damage has been described in a model of concanavalin A immune-mediated hepatitis. These data further support a role for activated regulatory cells in the process of AVC.

The present invention demonstrates that administration of β-glycosphingolipids decreases cleaved caspase 3 in treated valves. Chronic apoptosis was recently shown to accelerate atherosclerosis and promotes calcification and medial degeneration. It was suggested that apoptosis precedes the calcification process and apoptotic bodies induce calcification. Activation of cardiomyocyte caspase enzymes occurs during the transition to heart failure in patients with aortic stenosis. It was suggested that apoptosis-based strategies may slow the progression of heart failure in aortic stenosis patients.

NKT cells have been implicated in the regulation of adaptive immune responses, including those directed against autoantigens. Furthermore, abnormalities in the number and function of NKT cells have been observed in patients with autoimmune diseases and in a variety of mouse strains genetically predisposed for the development of autoimmune diseases. CD1d-restricted NKT cells exacerbate atherosclerosis and are considered pro-atherogenic. Adoptive transfer of splenocytes enriched in a restricted population of NKT cells enhances atherosclerosis in the aortic root compared with those lacking NKT cells. However, no studies have determined the role for NKT cells in AVC, and the present data support a role for NKT lymphocytes in the inflammatory process associated with valve calcification in AVC.

β-glycosphingolipids, are naturally occurring glycolipids with heterogeneous mixture of long N-acyl chains on the ceramide backbone. In several murine models, their immune modulatory effect was associated with altered CD8 and NKT distribution. The beneficial effect of β-glycosphingolipids was shown by the present invention as associated with altered CD8 and NKT lymphocyte distribution pattern. Treated animals exhibited a similar pattern to the one observed in normal animals, further supporting a functional role for CD8 and NKT in the pathogenesis of AVC, and an ability of β-glycosphingolipids to alter NKT function.

The results of the present invention support a role for the RANK pathway-dependent inflammation in the development of CAS. NKT and CD8 lymphocytes may mediate the immune process associated with the calcification process. β-glycosphingolipids, by decreasing RANK expression and inhibiting the accumulation of NKT and CD8 cells in aortic valves, significantly alleviated the calcification. These data further support a reversibility of the immune-mediated calcification in AVC. β-glycosphingolipids were recently shown to be safe and potentially effective in humans, thus suggesting that they may be effective in the treatment of patients with AVC.

Moreover, the invention further demonstrated the reversibility of AVC using phosphate inhibitors or any compound that alters the phosphate binding or transporting into or out of any cell or any membrane or any combination thereof. More specifically, the invention shows that inhibition of PIT1, using foscarnet, resulted in alleviation of the calcification process.

The data of the present invention therefore provides a comprehensive approach combining both, the immunomodulatory potential of β-glycolipids and the calcification inhibitory effect of phosphate inhibitors.

Thus as a first aspect, the present invention relates to a composition comprising a combination of at least one natural or synthetic β-glycolipid, a mixture of at least two β-glycolipids, a substance which increases the intracellular, extracellular or serum level of a naturally occurring β-glycolipid, or any combination or mixture thereof and at least one phosphate inhibitor or any compound that alters the phosphate binding or transporting into or out of any cell or any membrane or any combination thereof. It should be noted that the composition of the invention may optionally further comprises at least one pharmaceutically acceptable carrier, diluent, excipient and/or additive.

According to one embodiment, the β-glycolipid comprised within the combined composition of the invention may be selected from the group consisting of a lactosyl-ceramide, a glucosylceramide, a monosaccharide ceramide, a galatosylceremide, a gal-gal-glucosyl-ceramide, GM2 ganglioside, GM3 ganglioside, globoside or any other β-glycolipid, any natural or synthetic analogs derivatives thereof and any combinations or mixtures thereof.

According to one specific embodiment, which is in no way limiting, synthetic derivatives of β-glycolipids may be used for the combined composition of the invention. More particularly, a compound of formula I, or isomer or a pharmaceutically acceptable salt thereof may be a preferable derivative.

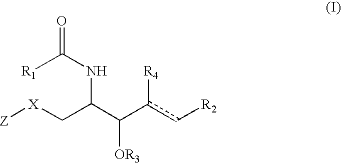

Preferably, wherein R1 is C₅₋₂₅alkyl optionally substituted with up to two substituents selected from OH, F, Cl, Br, and optionally comprising up to 4 non-adjacent and non-conjugated double bonds;

R2 is C₇₋₂₁ alkyl optionally comprising up to 4 non-adjacent and non-conjugated double bonds;

R3 is H, C₁₋₆alkyl, C₁₋₆acyl,H₂PO₃, or HSO₃;

R4, is H or OH;

X is O or S;

Z is an optionally branched oligosaccharide comprising from 1 to 6 hexose units selected from glucose (Glc), galactose (Gal), N-acetyl-galactosamine (GalNAc), fucose (Fuc), N-acetyl-neuraminic acid (NeuNAc), wherein at least one free hydroxyl group of said hexose units may be optionally substituted with O—C₁₋₆alkyl, O—C₁₋₆acyl, O-benzyl, O—H2PO₃, or O—HSO₃; and wherein [symbol] symbolizes either double or single bond; with the proviso that' when X is O, and R₃ is H, and R₂ is unsaturated unbranched alkyl C₁₃, then R₁ is not unsubstituted unsaturated, unbranched alkyl.

In yet another embodiment, the combined composition of the invention may use the compound of Formula II, or isomer thereof or a pharmaceutically acceptable salt thereof as a β-glycolipid derivative.

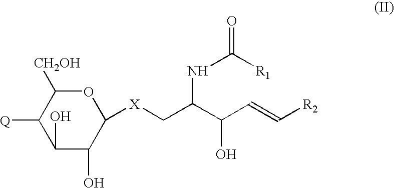

According to this embodiment, R1 is C₅₋₂₅alkyl optionally comprising up to 4 non-adjacent and non-conjugated double bonds;

R2 is C₇₋₂₁alkyl;

X is O or S;

Q is OH in any anomeric configuration or Gal, thus wherein at least one free hydroxyl group of the monosaccharide units may be optionally substituted with O—C₁₋₆alkyl, O—C₁₋₆ acyl, O-benzyl, O—H2O₃, or O—HSO₃. It should be noted that the formula of the compound of the invention is with the proviso that when X is O, and R₂ is unsaturated unbranched alkyl C₁₃, then R₁ is not unsubstituted unsaturated unbranched alkyl.

Still further, according to another non-limiting embodiment, the combined composition of the invention may comprises as a β-glycolipid derivative, the compound of formula I or isomer thereof or a pharmaceutically acceptable salt thereof, wherein Z is selected from Glc, Gal, and lactose.

In yet another preferred embodiment, which is in no way limiting, the compound of formula III, or a pharmaceutically acceptable salt thereof may be used as a β-glycolipid derivative for the combined composition of the invention.

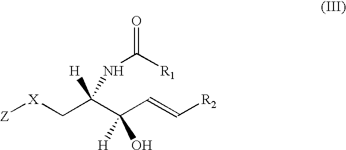

According to this particular embodiment, wherein R1 is C₅₋₂₅alkyl optionally substituted with up to two substituents selected from OH, F, Cl, Br, and optionally comprising up to 4 non-adjacent and non-conjugated double bonds; R2 is C₇₋₂₁alkyl optionally comprising up to 4 non-adjacent and non-conjugated double bonds; X is O or S;

Z may be selected from β-glucose, β-galactose, and β-lactose, wherein at least one free hydroxyl group of said Glc, Gal, or lactose may be optionally substituted with O—C₁₋₆alkyl, O—C₁₋₆ acyl, O-benzyl, O—H₂PO₃, or O—HSO₃. It should be appreciated that the compound of Formula III may be with the proviso that when X is O, and R₂ may be unsaturated unbranched alkyl C₁₃, then R₁ is not unsubstituted unsaturated unbranched alkyl.

Compounds of formula I according to the invention are active as homologues of glycosylceramides. Included in the invention are also isomers of the compounds of formula I, including optical isomers, cis-trans isomers, anomers, etc., as well as their mixtures. R₁ in formula I may be an alkyl having from 5 to 25 carbon atoms, preferably an unbranched C₅₋₂₅ alkyl aliphatic chain, saturated or unsaturated, optionally substituted with up to two substituents selected from OH, F, Cl, Br, comprising up to 4 non-adjacent and non-conjugated double bonds; said alkyl is preferably C₁₃₋₂₅alkyl. R₂ in formula I is an alkyl having from 7 to 21 carbon atoms, preferably an unbranched C₇₋₂₁alkyl aliphatic chain, saturated or unsaturated, comprising up to 4 non-adjacent and non-conjugated double bonds; said alkyl is preferably C₁₃₋₂₁alkyl. R₃ in formula I may be H, C₁₋₆alkyl, C₁₋₆ acyl, or other groups, R₄ is H or OH, and X is O or S. Z in formula I represents an optionally branched oligosaccharide comprising 6 hexose, units selected preferably from glucose (Glc), galactose (Gal), N-acetyl-galactosamine (GalNAc), fucose (Fuc), N-acetyl-neuraminic acid (NeuNAc). Z may be, for example, Glc-, Gal-, Glc-Gal-, or Glc-Gal-Gal-; the oligosaccharide configuration may comprise lactose, globoside, ganglioside saccharide Gₘ₂, ganglioside saccharide G_{M4}, etc. The free hydroxyl group of said hexose units may be substituted, preferably with O—C₁₋₆alkyl, O—C₁₋₆acyl, or O-benzyl. Z may comprise, for example, 3-O-acyl-Glc, 4-O-acyl-Gal, 6-O-acyl-Glc, etc. Said homologues of glycosylceramides according to the invention may be neutral, or may be rendered electrically charged, for example by substituting hydroxyl groups with —O—H2PO₃, or —O—-HSO₃.

Thus, the compounds of any one of Formulas I, II and III may be used by the combined composition of the invention as preferred synthetic derivatives of β-glycolipids.

As indicated above, the combined composition of the invention encompasses the use of β-glycolipid and any natural or synthetic analogs and derivatives thereof. Synthetic derivatives may include β-glycolipid modified by alteration of the acyl chain via elongation or truncation of the chain to any final number of carbons. Such derivative may alternatively or additionally include alteration of the acyl chain by adding a number of double bonds. In yet another embodiment, the β-glycolipid used by the combined composition of the invention may be modified by alteration of the sphingosine chain by replacing it with any possible lipid chain. Addition of thiol group between the sugar moiety and any of the chains or addition of any type of a ring to the molecule is also encompassed by the invention. It should be appreciated that a derivative according to the invention may include any combination of these modifications.

It should be appreciated that a combined composition comprising a β-glycolipid other than glucosylceramide is also contemplated within the scope of the invention. Therefore, according to a particular embodiment, the combined composition of the invention may comprise any β-glycolipid other than glucosylceramide.

According to a specifically preferred embodiment the β-glycolipids used by the combined composition of the invention may be β-lactosyl-ceramide and any analogue or derivative thereof.

According to another specifically preferred embodiment the β-glycolipids used by the combined composition of the invention may be β-glucosylceramide and any analogue or derivative thereof.

In yet another preferred embodiment, a mixture of β-glycolipids comprised within the combined composition of the invention may contain at least two β-glycolipids at a quantitative ratio between 1:1 to 1:1000. It should be appreciated that any quantitative ratio may be used. As a non-limiting example, a quantitative ratio used may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the mixture contained within the combined composition of the invention comprises more than two glycolipids, the quantitative ratio used may be for example, 1:1:1, 1:2:3, 1:10:100, 1:10:100:1000 etc.

According to a specifically preferred embodiment, the combined composition of the invention comprises a mixture of preferred β-glycolipids. More specifically, such mixture may comprise β-lactosyl-ceramide and at least one other β-glycolipid at a quantitative ratio between any one of 1:1 to 1:1000 or 1:1 to 1000:1. More preferably, such mixture comprises β-glucosylceramide (GC) and β-lactosyl-ceramide (LacC) at a quantitative ratio between any one of 1:1 to 1:1000 and 1:1 to 1000:1. Most preferably, the mixture used by the combined composition of the invention may comprise β-glucosylceramide (GC) and β-lactosyl-ceramide (LacC) at a quantitative ratio of any one of 1:1 and 1:1000, preferably, 1:1.

Based on previous results of the present inventors which demonstrate the use of mixtures of β-glycolipids for treating immune-related disorders (WO 2007/060652), a daily amount of such preferred mixtures, may contain between about 0.01 to 50, preferably, 0.5 to 5 mg per kg of body weight of β-glucosylceramide and between about 0.1 to 500, preferably, 5 to 50 mg per kg of body weight of β-lactosyl-ceramide at a quantitative ratio of 1:1 to 1:1000, preferably of 1:1.

The results of the present invention indicate that an amount of 2.5 mg/kg of the IGL mixture (GC and LacC 1:1) exhibit a beneficial effect on degree of aortic stenosis as well as aortic valve inflammatory markers. Therefore, according to one embodiment, the mixture used for the combined composition of the invention may comprise between about 0.5 to 10 mg per kg of body weight of β-glucosylceramide and between about 1 to 50 mg per kg of body weight of β-lactosyl-ceramide at a quantitative ratio of between about 1:1 to 1:10. According to one specifically preferred embodiment, the β-glycolipid mixture used by the combined composition of the invention comprises between about 0.5 to 10 mg per kg of body weight of β-glucosylceramide and β-lactosyl-ceramide mixture, for example, 0.75, 1, 1.5, 2, 2.5, 3, 3.5, 4, 4.5, 5, 5.5, 6, 6.5, 7, 7.5, 8, 8.5, 9, 9.5 and 10 mg per kg of body weight of β-glucosylceramide and β-lactosyl-ceramide mixture. It should be noted that any effective amount may be also used. According to a particular embodiment, the mixture used by the invention comprises between about 2.5mg per kg of body weight of β-glucosylceramide and β-lactosyl-ceramide mixture at a quantitative ratio of 1:1. It should be appreciated that such mixture of β-glucosylceramide and β-lactosyl-ceramide at a quantitative ratio of 1:1, is referred to by the invention as IGL.

It should be noted that a substance which increases the intracellular, extracellular or serum level of a naturally occurring β-glycolipid, may increase the rate of production of said β-glycolipid in a treated subject, or decrease the rate of degradation or turnover of said β-glycolipid in said subject.

According to a specifically preferred embodiment, the combined composition of the invention comprises a phosphate inhibitor or any compound that alters the phosphate binding or transporting into or out of any cell or any membrane or any combination thereof. The phosphate inhibitor used by the combined composition of the invention may be any compound or substance having a modulatory effect on phosphate levels or metabolism. Such phosphate inhibitor may be for example, a sodium-phosphate co transporter PIT 1 inhibitor, a phosphate inhibitor and a Parathyroid hormone (PTH) inhibitor.

As shown by Example 6, the inventors used the PIT 1 inhibitor, foscarnet, for inhibiting valve calcification. Thus, according to a particular embodiment, the PIT 1 inhibitor used by the combined composition of the invention may be foscarnet. Foscarnet is the conjugate base of the chemical compound with the formula HO₂CPO₃H₂. This phosphonic acid derivative (marketed by AstraZeneca as foscarnet sodium under the trade name Foscavir) is an antiviral medication used to treat herpes viruses, including cytomegalovirus (CMV) and herpes simplex viruses types 1 and 2 (HSV-1 and HSV-2). It is particularly used to treat CMV retinitis.

Foscarnet is a structural mimic of the anion pyrophosphate that selectively inhibits the pyrophosphate binding site on viral DNA polymerases at concentrations that do not affect human DNA polymerases.

The results of the present invention indicate that an amount of 5 mg/kg of the PIT 1 inhibitor foscarnet, administered i.p., exhibited a beneficial effect on degree of aortic stenosis as well as aortic valve inflammatory markers. Therefore, according to one embodiment, the combined composition of the invention may comprise between about 0.5 to 10 mg per kg of body weight of foscarnet. According to one specifically preferred embodiment, the combined composition of the invention may comprises any one of 0.75, 1, 1.5, 2, 2.5, 3, 3.5, 4, 4.5, 5, 5.5, 6, 6.5, 7, 7.5, 8, 8.5, 9, 9.5 and 10 mg per kg of body weight of foscarnet. It should be noted that any effective amount may be also used. According to a particular embodiment, the combined composition of the invention may comprise about 0.5 mg/kg of body weight of foscarnet.

According to another preferred embodiment, the combined composition of the invention may comprises a β-glycolipid, or any mixture thereof and a phosphate inhibitor or any compound that alters the phosphate binding or transporting into or out of any cell or any membrane or any combination thereof, at a quantitative ratio between about any one of 1:1 to 1:1000 and 1:1 to 1000:1. A particular composition may comprise at least one β-glycolipid, preferably the IGL combination and foscarnet.

According to one embodiment, the combined composition of the invention may be a therapeutic composition particularly useful for the treatment of a calcification related degenerative disorder, such as any one of a vascular or valvular degenerative disorder and calcification related disorders in visceral organs.

Thus, according to another aspect, the invention relates to a therapeutic composition for the treatment of a calcification related degenerative disorder in a mammalian subject. According to one embodiment, the composition of the invention may comprise as an active ingredient, at least one natural or synthetic β-glycolipid, a mixture of at least two β-glycolipids, a substance which increases the intracellular, extracellular or serum level of a naturally occurring β-glycolipid, or any combination or mixture thereof and at least one phosphate inhibitor or any compound that alters the phosphate binding or transporting into or out of any cell or any membrane or any combination thereof.

According to one embodiment, calcification related degenerative disorder may be any one of a vascular or valvular degenerative disorder and calcification related disorders in visceral organs.

In another embodiment, the composition of the invention may optionally further comprises at least one other active ingredient/s that improve the therapeutic effect, whether administered in combination, serially or simultaneously.

The term “Calcification”, refers to a process of mineralization of soft tissue in which calcium is deposited in otherwise normal tissue, because of elevated levels of calcium in blood because of deranged metabolism, synthesis or disposal. However, it should be noted that calcification may also appear with normal levels of calcium due to other mechanisms. As used here the term is intended to mean the abnormal deposition of calcium crystals at sites other than bones and teeth. Calcification results in the accumulation of macroscopic amorphous calcium phosphate and hydroxyapatite deposits in the extracellular matrix that may lead to a number of clinical conditions. Calcification can occur in a variety of tissues and organs commonly, in vascular tissue, including arteries, veins, capillaries valves and sinuses but also, in non-vascular tissues, such as tendons, skin, sclera and myometrium.

Therefore, according to a specific preferred embodiment the calcification related degenerative disorder may be a vascular or valvular degenerative disorder. Calcification is one possible stage of atherosclerosis, when calcium deposits collect on growing atherosclerotic plaques, but calcification is also common in visceral organs, including the lung, kidney and stomach, brain, pancreas and in diseases resulting from mineral imbalance; such as renal failure and diabetes.

Examples of degenerative cardiovascular diseases that feature or result from calcification include Arteriosclerosis, Atherosclerosis, Angina, Cardiomyopathy, Arrhythmia, Arterial aneurysms, Congestive heart failure, Coronary artery disease, Thromboembolism and Cerebrovasculal diseases such as Aneurysms, Cerebral embolisms, Transient cerebral ischemia and Stroke.

According to one specific embodiment, the combined therapeutic composition of the invention is particularly intended for treating AVC (aortic valve calcification).

In yet another embodiment, the invention provides a combined therapeutic composition for treating aortic stenosis (AS). Moreover, it should be appreciated that the combined composition of the invention may be, used for the treatment of any disorder or condition “associated with”, “linked to”, “caused by” any calcification degenerative disorder, in a non-limiting example, the composition of the invention may be used for treating renal failure in a subject in need thereof.

The combined composition of the invention should be applied to a subject suffering from a pathologic disorder such as an atherosclerotic cardiovascular disease, an atherosclerotic peripheral vascular disease, peripheral venous disease, a calcific valvular stenosis, calcific valvular regurgitation and prosthetic or biologic artificial valve's calcification or dysfunction.

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

“Vascular disorders” and “valvular disorders” are diseases or disorders characterized by an abnormality or malfunction of the vasculature or valves. Vascular disorders can affect the system of blood vessels known as the circulatory system which supply oxygen-rich blood from the heart and remove waste products from the cells. Valvular disorders can effect the normal functioning of the heart valves system, which allows the unidirectional flow of blood at the right time. It should be noted that as used herein, valvular diseases include a calcific valvular stenosis, calcific valvular regurgitation and prosthetic or biologic artificial valve's calcification or dysfunction.

Vascular disorders include any condition that affects the circulatory system. While the term technically refers to any disease that affects the arteries, veins and lymph nodes, it is usually used to refer to diseases that involve the heart or blood vessels (arteries and veins) (e.g. cardiovascular diseases). Therefore, vascular disorders include an atherosclerotic cardiovascular disease, an atherosclerotic peripheral vascular disease and a peripheral venous disease. The common denominator at the base of many and principal vascular diseases and particularly peripheral artery diseases such as aortic stenosis, coronary heart disease, carotid artery disease, transient cerebral ischemia, peripheral artery disease of the lower extremities (legs), renal artery disease, abdominal aortic aneurysm, Raynaud's phenomenon (also called Raynaud's disease or syndrome), as well as Buerger's disease and Polyarteritis nodosa, was found to be the narrowing and hardening of one or more artery, a from of atherosclerosis.

It should be noted that peripheral artery diseases as used herein include vasculitis of all sizes of blood vessels: small (e.g. Henoch-Shonlein purpura), medium (e.g Chrung Struss syndrome), large (e.g. Takayasu disease).

“Vascular degenerative disorders” and “valvular degenerative disorders” are complex and pernicious diseases, whose onset is insidious, followed by progressive deterioration. Clinical manifestations are determined by the location and seriousness of the disorder. Vascular and valvular degenerative diseases remain asymptomatic for the most part of their progression until an acute event develops in later life. Symptomatic patients may exhibit dizziness, syncope, angina, head pressure, severe headaches, and loss of control over involuntary muscles, facial weakness, slurred speech, vision loss, and numbness within limbs, loss of coordination and the ability to walk. Exemplary vascular and valvular degenerative diseases include cardiovascular diseases, peripheral vascular diseases and valvular heart diseases.

The term “Cardiovascular disease”, refers to the class of diseases that involve the heart or blood vessels (arteries and veins). While the term technically refers to any disease that affects the cardiovascular system, it is usually used to refer to those related to atherosclerosis (arterial disease).

The term “Atherosclerosis”, refers to a cardiovascular disease affecting arterial blood vessels. It is a chronic inflammatory response in the walls of arteries, in large part due to the deposition of lipoproteins. It is commonly referred to as a “hardening” or “furring” of the arteries. It is caused by the formation of multiple atheromatous plaques (an accumulation and swelling in artery wells) within the arteries. The plaques, created due to injury of the cells of the endothelial lining which is followed by inflammation, are made of the accumulation of macrophages, penetrating cholesterol molecules that further exacerbate the injury and inflammation and, of a calcium deposit (calcification) at the outer base of older or more advanced lesions. The plaque eventually leads to ruptures and stenosis (narrowing) of the artery and, therefore, an insufficient blood supply (ischemia) to the organ it feeds. Alternatively, overcompensation by excessive enlargement of the artery leads to aneurysm.

“Peripheral vascular diseases” often called “Peripheral artery diseases”, is a group of diseases caused by the obstruction of large peripheral arteries, which can result from atherosclerosis, inflammatory process leading to stenosis, an embolism or thrombus formation. It causes either acute or chronic ischemia. Patients suffering from peripheral artery diseases exhibit intermittent claudication and may develop critical (chronic) limb ischemia with rest pain, non-healing ulcers, and/or gangrene. When left untreated critical limb ischemia may develop into an acute limb ischemia.

Peripheral artery diseases include Carotid artery disease, Peripheral artery disease of the lower extremities (legs), Peripheral artery disease of renal arteries, Raynaud's phenomenon (also called Raynaud's disease or syndrome), Buerger's disease and. Polyarteritis nodosa and vasculitis of all sizes of blood vessels: small (e.g Henoch-Shonlein purpura), medium (e.g Chrung Struss syndrome), large (e.g Takayasu disease).

“Valvular heart diseases” is the name given to any dysfunction or abnormality of one or more of the heart's four valves. The most common type is valvular stenosis. Valvular stenosis is a condition in which there is a narrowing, stiffening, thickening, fusion or blockage of one or more of the valves of the heart. Mild valvular stenosis may not show any symptoms, but as stenosis worsen, symptoms of dizziness, syncope, angina, damage to the myocardium, left ventricular hypertrophy, valvular regurgitation, arrhythmia and congestive heart failure may develop. Valvular stenosis may develop before birth or may be acquired after birth as a result of conditions such as rheumatic fever and calcification of the leaflets of the heart valve as indicated herein before, valvular diseases as used herein include prosthetic or biologic artificial valve's calcification or dysfunction. More particularly, the invention further encompasses combined compositions for the treatment of disorders involving calcification of mechanical valves, whether biological or synthetic, as well as any type of calcification of any stent.

Valvular heart diseases include Aortic stenosis, Mitral stenosis, Tricuspid stenosis and Pulmonary stenosis. The most common type being Aortic stenosis. In another embodiment, valvular diseases include calcific valvular regurgitation. Valvular regurgitation, also known as valvular incompetence or valvular insufficiency, is a condition in which blood leaks in the wrong direction because one or more of the heart's valves is closing improperly. Valvular regurgitation may occur in any of the four valves of the heart, the aortic valve, the mitral valve, the tricuspid valve or the pulmonic valve. It is assumed that there is normally no flow backwards into the ventricles through the aortic or pulmonic valves in diastole. Similarly, there is no flow backwards into the atria across the mitral or tricuspid valves in systole. Thus, the first effect of regurgitation on blood flow through the heart is a change in direction. The second effect of regurgitation on cardiac blood flow is the creation of turbulence. Regurgitation originates from small, irregular openings. They may be directed quite eccentrically and they are almost always turbulent, they are made up of many different velocities and complex flow patterns.

In yet another embodiment, the combined composition of the invention may be applicable for the treatment of peripheral venous diseases, such as varicose veins and chronic venous, insufficiency.

As indicated herein before, it should be appreciated that the combined composition of the invention may be applicable also for the treatment of non-vascular calcification-related degenerative disorders. More specifically, the combined composition of the invention may be used for treating calcification related disorders in visceral organs. Examples for such disorders may be Kidney and Bladder stones, Gall Stones, Pancreas and Bowel diseases (such as Pancreatic duct stones, Calcific Pancreatitis, Crohn's disease, Colitis ulcerosa), Liver diseases (such as Liver cirrhosis, Liver cysts), Prostate calcification, Type 1 Diabetes mellitus, Eye diseases (such as Corneal calcifications, Cataracts, Macular degeneration, Retinal nerve degeneration, Retinitis, Iritis), Ear diseases (such as Otosclerosis, Degeneration of Otolithus) Skin diseases (such as Calcinosis Cutis, Calciphylaxis, Eczema, Psoriasis), Rheumatic arthritis, Calcific Tenditis, Splenic calcifications, Chronic Obstructive Pulmonary disease, Broncholiths, Bronchial stones, Calcifications and Encrustations of implants.

The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein, fully incorporated herein by reference.

The pharmaceutical composition of the invention can be administered and dosed in accordance with good medical practice. Administration may be carried out in various ways, including intravenous, intraperitoneal, intramuscular or subcutaneous injection. However, other methods of administration such as nasal or oral administration may be preferred.

The composition of the invention may comprise the active substance in free form and be administered directly to the subject to be treated. Formulations typically comprise at least one active ingredient, as defined above, together with one or more acceptable carriers thereof. Each carrier should be both pharmaceutically and physiologically acceptable in the sense of being compatible with the other ingredients and not injurious to the patient.

Formulations include those suitable for oral, nasal, or parenteral (including subcutaneous (s.c.), intramuscular (i.m.), intraperitoneal (i.p.), intravenous (i.v.) and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringe ability exists. The compositions must be stable under the conditions of manufacture and storage and must be preserved against the contaminating action of microorganisms, such as bacteria and fungi.

In many cases, it will be preferable to include isotonic agents, for example, sugars or sodium chloride. Prolonged absorption of the injectable compositions can be brought about by the use in the compositions of agents delaying absorption.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

The pharmaceutical compositions of the invention generally comprise a buffering agent, an agent that adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

In instances in which oral administration is in the form of a tablet or capsule, the active drug components can be combined with a non-toxic pharmaceutically acceptable inert carrier such as lactose, starch, sucrose, glucose, modified sugars, modified starches, methylcellulose and its derivatives, dicalcium phosphate, calcium sulfate, mannitol, sorbitol, and other reducing and non-reducing sugars, magnesium stearate, stearic acid, sodium stearyl fumarate, glyceryl behenate, calcium stearate and the like. For oral administration in liquid form, the active drug components can be combined with non-toxic pharmaceutically acceptable inert carriers such as ethanol, glycerol, water and the like. When desired or required, suitable binders, lubricants, disintegrating agents and coloring and flavoring agents can also be incorporated into the mixture. Stabilizing agents such as antioxidants, propyl gallate, sodium ascorbate, citric acid, calcium metabisulphite, hydroquinone, and 7-hydroxycoumarin can also be added to stabilize the dosage forms. Other suitable compounds can include gelatin, sweeteners, natural and synthetic gums such as acacia, tragacanth, or alginates, carboxymethylcellulose, polyethylene, glycol, waxes and the like.

Alternatively, the composition of this invention may also be administered in controlled release formulations such as a slow release or a fast release formulation. Such controlled release formulations of the combination of this invention may be prepared using methods well known to those skilled in the art. The method of administration will be determined by the attendant physician or other person skilled in the art after an evaluation of the subject's conditions and requirements.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art methods of preparing various pharmaceutical compositions with a certain amount of active ingredient are known, or will be apparent in light of this disclosure, to those skilled in this art.

It should be appreciated that the composition of the invention which may comprise a combination of at least one natural or synthetic β-glycolipid, a mixture of at least two β-glycolipids, a substance which increases the intracellular, extracellular or serum level of a naturally occurring β-glycolipid, or any combination or mixture thereof and at least one phosphate inhibitor or any compound that alters the phosphate binding or transporting into or out of any cell or any membrane or any combination thereof, may preferably exhibit a synergetic effect. By synergic combination is meant that the effect, of both active ingredients, for example, a β-glycolipid, or a mixture thereof, specifically, the IGL, and the phosphate inhibitor, specifically, foscarnet, is greater than the sum of the therapeutic effects of administration of any of these compounds separately, as a sole treatment.

The present invention relates also to the treatment of diseases and conditions with a combination of different β-glycolipids (or a mixture thereof) or combinations of β-glycolipids with other active ingredients, specifically, with at least one phosphate inhibitor or any compound that alters the phosphate binding or transporting into or out of any cell or any membrane or any combination thereof. It should be noted that all possible combined active ingredients may be administered separately. Therefore, the invention also provides combination of separate pharmaceutical compositions in kit form.

Thus, according to a further aspect, the invention relates to a kit for achieving a therapeutic effect in a subject in need thereof, specifically for treating calcification related degenerative disorder. According to this aspect, the kit of the invention comprises: (a) at least one natural or synthetic β-glycolipid, a mixture of at least two β-glycolipids, a substance which increases the intracellular, extracellular or serum level of a naturally occurring β-glycolipid; or any combination or mixture thereof, or a pharmaceutically acceptable derivative thereof and a pharmaceutically acceptable carrier or diluent, optionally, in a first unit dosage form; (b) at least one phosphate inhibitor any compound that alters the phosphate binding or transporting into or out of any cell or any membrane or any combination thereof and a pharmaceutically-acceptable carrier or diluent, optionally, in a second unit dosage form; and (c) container means for containing said first and second dosage forms.

More specifically, the kit includes two separate pharmaceutical compositions, for example, at least one β-glycolipids and at least one phosphate inhibitor.

The kit may include container means for containing both separate compositions, such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

According to one embodiment the kit of the invention is intended for achieving a therapeutic effect in a subject suffering from any vascular or valvular degenerative disorder associated with blockage of a blood vessel and/or calcification.

Still further, the invention provides a method of treatment of a pathologic disorder comprising the step of administering to a subject in need thereof a therapeutically effective amount of a first and a second unit dosage forms comprised in the kit according to the invention.

It should be appreciated that both components of the kit, the certain β-glycolipid or a combination of at least two β-glycolipids, preferably, in the first dosage form and the at least one phosphate inhibitor, preferably, in the-second dosage form may be administered simultaneously.

Alternatively, said first compound or dosage form and said second compound or dosage form are administered sequentially in either order.

The inventors speculate that the mechanism by which β-glycolipids exert their mollifying effect upon vascular and valvular degenerative -disorders is performed by influencing the inflammatory pathway leading to atherosclerosis as well as by affecting the calcification process.

A further aspect of the invention relates to a method for the treatment or prevention of a calcification related degenerative disorder, in a subject in need thereof. According to one embodiment, the method of the invention comprises the step of administering to said subject a therapeutically effective amount of at least one of: (a) at least one of a natural or synthetic β-glycolipid, a mixture of at least two β-glycolipids, a substance which increases the intracellular, extracellular or serum level of a naturally occurring β-glycolipid; (b) at least one phosphate inhibitor or any compound that alters the phosphate binding or transporting into or out of any cell or any membrane or any combination thereof; and (c) a composition or kit comprising (a) or (b) or of any combinations thereof.

According to another preferred embodiment, the β-glycolipid used by the method of the invention may be selected from the group consisting of a monosaccharide ceramide, a glucosylceramide, a galatosylceremide, a lactosyl-ceramide, a gal-gal-glucosyl-ceramide, GM2 ganglioside, GM3 ganglioside, globoside or any other β-glycolipid. Preferably, the β-glycolipid used by the composition of the invention may be β-latosyl-ceremide and any analogue or derivative thereof. The use of a β-glycolipid other than glucosylceramide is also within the scope of the invention.

In yet another preferred embodiment, a mixture of β-glycolipids used by the method of the invention may comprise at least two β-glycolipids at a quantitative ratio between 1:1 to 1:1000. It should be appreciated that any quantitative ratio may be used.

According to a specifically preferred embodiment, a mixture of preferred β-glycolipids used by the method of the invention may comprise β-lactosyl-ceramide and at least one other β-glycolipid at a quantitative ratio between 1:1 to 1:1000. More preferably, such mixture comprises β-lactosyl-ceramide and β-glucosylceramide at a quantitative ratio between 1:1 to 1:1000. Most preferably, the mixture used the method of the invention may comprise β-lactosyl-ceramide (LacC) and β-glucosylceramide (GC) at a quantitative ratio of any one of 1:1 and 1:1000, preferably, 1:1.

Different combinations of different ratios at different concentrations of GC and LacC may be used for a method useful for treating different vascular and valvular degenerative disorders. A daily dose of the active ingredients in a preferred mixture, may contain between about 0.01 to 50, preferably, 0.5 to 10 mg per kg of body weight of β-glucosylceramide (GC) and between about 0.1 to 500, preferably, 1 to 50 mg per kg of body weight of β-lactosyl-ceramide (LacC) at a quantitative ratio of 1:1 of 1:1000, preferably of 1:1.

According to one specific embodiment, the mixture used by the method of the invention may comprise between about 0.5 to 10, preferably, 2.5 mg per kg of body weight of a mixture of β-glucosylceramide and β-lactosyl-ceramide at a quantitative ratio of 1:1

According to another embodiment, the phosphate inhibitor used for the method of the invention may be any one of a sodium-phosphate co transporter PIT 1 inhibitor, a phosphate inhibitor and a Parathyroid hormone (PTH) inhibitor. More specifically, such PIT 1 inhibitor may be foscarnet.

It should be noted that the method of the invention may use any of the combined compositions of the invention.

According to one embodiment, the method of the invention is particularly applicable for the treatment of a calcification related degenerative disorder, for example, any one of a vascular or valvular degenerative disorder and calcification related disorders in visceral organs. According to a specifically preferred embodiment, the method of the invention is intended for treating vascular and valvular degenerative disorders. More specifically, vascular or valvular degenerative disorder may be any one of an atherosclerotic cardiovascular disease, an atherosclerotic peripheral vascular disease, peripheral venous disease, a calcific valvular stenosis, calcific valvular regurgitation and prosthetic or biologic artificial valve's calcification or dysfunction.

More specifically, a vascular or valvular degenerative disorder may be for example: any one of the group of atherosclerotic cardiovascular disease consisting of Arteriosclerosis, Atherosclerosis, Cardiomyopathy, Arrhythmia, Arterial aneurysms, Congestive heart failure, Coronary artery disease, Cerebrovascular diseases (such as, Aneurysms, Cerebral embolisms, Transient cerebral ischemia; Stroke) and Thromboembolism. Peripheral vascular diseases as used herein (also known as Peripheral artery diseases) may include Carotid artery disease, Peripheral artery disease of the lower extremities (legs), Peripheral artery disease of renal arteries, Raynaud's phenomenon (also called Raynaud's disease or syndrome), Buerger's disease, Polyarteritis nodosa and vasculitis of all sizes of blood vessels: small (e.g Henoch-Shonlein purpura), medium (e.g. Chrung Struss syndrome), large (e.g. Takayasu disease).

According to another embodiment, the method of the invention may be used for the treatment of calcific valvular stenosis such as Aortic stenosis, Mitral stenosis, Tricuspid stenosis and Pulmonary stenosis. Moreover, the method of the invention may be used for treating calcific valvular regurgitation for example, Aortic regurgitation, Mitral regurgitation, Tricuspid regurgitation and Pulmonary regurgitation.

Still further, the invention provides a composition for treating peripheral venous disease such as Varicose veins and Chronic venous insufficiency.

According to another alternative embodiment, the method of the invention may be applicable also for the treatment of a non-vascular calcification-related degenerative disorders, for example, Kidney and Bladder stones, Gall Stones, Pancreas and Bowel diseases (such as Pancreatic duct stones, calcific pancreatitis, Crohn's disease, Colitis ulcerosa), Liver diseases (such as Liver cirrhosis, Liver cysts), Prostate calcification, Type 1 Diabetes mellitus, Eye diseases (such as Corneal calcifications, Cataracts, Macular degeneration, Retinal nerve degeneration, Retinitis, Iritis), Ear diseases(such as Otosclerosis, Degeneration of Otolithus) Skin diseases (such as Calcinosis Cutis, Calciphylaxis, Eczema, Psoriasis), Rheumatic arthritis, Calcific Tenditis, Splenic calcifications, Chronic Obstructive Pulmonary disease, Broncholiths, Bronchial stones, Calcifications and Encrustations of implants.

It should be appreciated that the method of the invention may use either β-glycolipids, phosphate inhibitors or a combination of both.

It should be appreciated that the preferred amounts of active ingredients are specific for a specific vascular or valvular degenerative disorder. Appropriate concentrations for any other vascular or valvular degenerative disorders should be determined by the treating physician.

According to one specific embodiment, the β-glycolipid used by the method of the invention, exert a protective effect upon the cells of the endothelial lining of the vasculature and valves, reducing both atherosclerotic and calcification processes.

A “protective effect” is aimed to prevent and treat complications that might result in vascular or valvular damage. The protection can be estimated by parameters of a calcium score, formation of an occlusion in a blood vessel or valve, arrest or slowing of the disease progression, disease onset and disease mortality delay.

According to another embodiment, the invention provides a method for prevention of the progression of vascular and valvular degenerative disease and is particularly applicable to patients suffering of chronic kidney disease or End stage renal disease.

More particularly, the above-mentioned possible effect of β-glycolipids on inflammatory and calcification processes may lead to clearance of the atheromatous plaque, and thereby alleviate vascular and valvular disorders involving the formation of plaques, plaque-like lesions or calcific occlusions of blood vessels and valves such as atherosclerosis, aortic sclerosis and aortic valve calcification.

Overall, it should be recognized that the effect of β-glycolipids may be mediated through alteration of function, intracellular machinery or distribution of components of the TNF signaling pathway, NKT lymphocyte, dendritic cell, any regulatory lymphocyte, and any type of lymphocyte, leading to an immune modulatory effect.

As indicated above, generally, the dosage of β-glycolipid needed to achieve a therapeutic -effect will depend not only on such factors as the age, weight and sex of the patient and mode of administration, but also on the degree of atheromatous plaque formation inhibition and calcification inhibition desired and the potency of the particular compound being utilized for the particular disorder of disease concerned.

In the case of vascular and valvular degenerative disorders, the subject amount is further characterized by its ability to inhibit or reduce plaque formation, as determined using in vitro assays or in vivo animal models of disease. For example in the case of Aortic valve calcification disease, the accumulation of inflammatory markers and the degree of valvular occlusion may be tested.

Experimental assays may include in vitro and in vivo experiments, based on the use of animal models, as presented in the invention.

According to a specifically preferred embodiment, the methods, kits and compositions of the invention are specifically suitable for the treatment of a mammalian subject. “Mammal” or “mammalian” for purposes of treatment refers to any animal classified as a mammal including, human, research animals, domestic and farm animals, and zoo, sports, or pet animals, such as dogs, horses, cats, cows, etc. In a particular embodiment said mammalian subject is a human subject.

The terms “treat, treating, treatment” as used herein and in the claims mean ameliorating one or more clinical indicia of disease activity in a patient having a calcification-related degenerative disease.

“Treatment” refers to therapeutic treatment. Those in need of treatment are mammalian subjects suffering from any vascular or valvular degenerative disorder By “patient” or “subject in need” is meant any mammal for which administration of β-glycolipid selected from the group consisting of a monosaccharide ceramide, a glucosylceramide, a galatosylceremide, a lactosyl-ceramide, a gal-gal-glucosyl-ceramide, GM2 ganglioside, GM3 ganglioside, globoside or any other β-glycolipid, or any natural or synthetic derivatives, or any mixture thereof, particularly, combination thereof with phosphate inhibitors or any pharmaceutical composition comprising the same is desired, in order to prevent, overcome, or slow down such infliction.

To provide a “preventive treatment” or “prophylactic treatment” is acting in a protective manner, to defend against or prevent something, especially a condition or disease.

As indicated above, the method of the invention is based on the administration of a therapeutically effective amount of at least one of: (a) at least one β-glycolipid selected from the group consisting of a monosaccharide ceramide, a glucosylceramide, a galatosylceremide, a lactosyl-ceramide, a gal-gal-glucosyl-ceramide, GM2 ganglioside, GM3 ganglioside, globoside or any other β-glycolipid and any natural or synthetic analogs and derivatives thereof and (b) at least one phosphate inhibitor or any compound that alters the phosphate binding or transporting into or out of any cell or any membrane or any combination thereof.

The terms “effective amount” or “sufficient amount” mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

According to another embodiment, the β-glycolipid used by the method of the invention or any mixture or combination thereof, or a combined composition of β-glycolipid and phosphate inhibitors may be administered alone, or in combination with other active ingredient/s that improve the therapeutic effect, whether administered in combination, serially or simultaneously.

According to another embodiment, the methods of the invention comprise administering to the treated subject an effective amount of at least one of:

(a) at least one β-glycolipid, a mixture of at least two β-glycolipids, a substance which increases the intracellular, extracellular or serum level of a naturally occurring β-glycolipid and any combination of the above and (b) at least one phosphate inhibitor or any compound that alters the phosphate binding or transporting into or out of any cell or any membrane or any combination thereof. According to this embodiment, the administering step comprises oral, intraperitoneal, intravenous, intramuscular, subcutaneous, perenteral, transdermal, intravaginal, intranasal, mucosal, sublingual, topical, rectal or subcutaneous administration, or any combination thereof.

Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

In a further aspect the invention relates to the use of a therapeutically effective amount of at least one of one of: (a) a β-glycolipid, a mixture of at least two β-glycolipids, and a substance which increases the intracellular, extracellular or serum level of a naturally occurring β-glycolipid or of any combinations and mixtures thereof; and (b) phosphate inhibitor or any compound that alters the phosphate binding or transporting into or out of any cell or any membrane or any combination or mixture thereof, in the preparation of a composition for the treatment of a calcification related degenerative disorder. According to one embodiment, calcification related degenerative disorder may be any one of a vascular or valvular degenerative disorder and calcification related disorders in visceral organs. According to a specific embodiment, the composition is as described by the invention.

According to one embodiment, the invention relates to the use of at least one β-glycolipid, a mixture of at least two β-glycolipids, and a substance which increases the intracellular, extracellular or serum level of a naturally occurring β-glycolipid or of any combinations and mixtures thereof), in the preparation of a composition for the treatment of a calcification related degenerative disorder. According to one embodiment, calcification related degenerative disorder may be any one of a vascular or valvular degenerative disorder and calcification related disorders in visceral organs. According to a specific embodiment, the composition is as described by the invention. According to one specific embodiment, the β-glycolipids used by the invention may be a mixture of β-glucosylceramide and β-lactosyl-ceramide, referred to by the invention as IGL.

According to an alternative embodiment, the present invention provides the use of phosphate inhibitor or any compound that alters the phosphate binding or transporting into or out of any cell or any membrane or any combination or mixture thereof, in the preparation of a composition for the treatment of a calcification related degenerative disorder. According to one embodiment, calcification related degenerative disorder may be any one of a vascular or valvular degenerative disorder and calcification related disorders in visceral organs. According to a specific embodiment, the composition is as described by the invention. Specifically, the invention relates to the use of PIT1 inhibitor, specifically, foscarnet, for the preparation of a composition for the treatment of a calcification related degenerative disorder.

According to another aspect, the invention provides a β-glycolipid selected from the group consisting of a lactosyl-ceramide, a glucosylceramide, a monosaccharide ceramide, a galatosylceremide, a gal-gal-glucosyl-ceramide, GM2 ganglioside, GM3 ganglioside, globoside or any other β-glycolipid, any natural or synthetic analogs derivatives thereof and any combinations or mixtures thereof for use in the treatment or prevention of a calcification related degenerative disorder. According to one specifically preferred embodiment, the invention provides β-lactosyl-ceramide or any combinations or mixtures thereof for the treatment or prevention of a calcification related degenerative disorder. According to another specifically preferred embodiment, the invention provides a β-glucosylceramide or any combinations or mixtures thereof for use in the treatment or prevention of a calcification related degenerative disorder.

In yet another embodiment, the invention provides a combination of β-glucosylceramide and β-lactosyl-ceramide, preferably, the IGL combination, for use in the treatment or prevention of a calcification related degenerative disorder. According to a specifically preferred embodiment, the invention provides a mixture comprising between about 0.5 to 10 mg per kg of body weight of β-glucosylceramide and between about 1 to 50 mg per kg of body weight of β-lactosyl-ceramide at a quantitative ratio of between 1:1 to 1:10, for use in the treatment or prevention of a calcification related degenerative disorder.

According to another specifically preferred embodiment, the invention provides at least one phosphate inhibitor or any compound that alters the phosphate binding or transporting into or out of any cell or any membrane or any combination thereof for use in the treatment or prevention of a calcification related degenerative disorder. More specifically, such phosphate inhibitor may be the PIT1 inhibitor, foscarnet.

According to one embodiment, such calcification related degenerative disorder may be any one of a vascular or valvular degenerative disorder and calcification related disorders in visceral organs.

In another aspect, the present invention relates to a method for protection of the cells of the endothelial lining from a degenerative process. It should be appreciated that a degenerative process as used herein may include apoptosis. The invention therefore provides a method for preventing or inhibiting apoptosis in aortic valve cells. It should be noted that as used herein “preventing” or “inhibiting” apoptosis may be reflected by reduction of between about 5, 10, 20, 30, 40, 50, 60, 70, 80, 90, 95 and 100% of apoptosis as compared to an appropriate control, for example, untreated cells or subject.

According to one embodiment, the degenerative process may be caused by inflammation and the formation of an atherosclerotic-plaque-related disorder in a mammalian subject. For example, an atherosclerotic cardiovascular disease, an atherosclerotic peripheral vascular disease, peripheral venous disease, a calcific valvular stenosis, calcific valvular regurgitation and prosthetic or biologic artificial valve's calcification or dysfunction.

More particularly, the vascular or valvular degenerative disorder may be any one of the group of atherosclerotic cardiovascular diseases consisting of Arteriosclerosis, Atherosclerosis, Cardiomyopathy, Arrhythmia, Arterial aneurysms, Congestive heart failure, Coronary artery disease, Cerebrovascular diseases (such as, Aneurysms, Cerebral embolisms, Transient cerebral ischemia, Stroke), and Thromboembolism.

The vascular or valvular degenerative disorder may be a Peripheral vascular disease (also known as Peripheral artery disease), for example, Carotid artery disease, Peripheral artery disease of the lower extremities (legs), Peripheral artery disease of renal arteries, Raynaud's phenomenon (also, called Raynaud's disease or syndrome), Buerger's disease, and Polyarteritis nodosa, as well as small medium or large vessel vasculitis.

The vascular or valvular degenerative disorder may be a calcific valvular stenosis, such as Aortic stenosis, Mitral stenosis, Pulmonic stenosis and. Tricuspid stenosis or any type of calcific valvular regurgitation, for example, Aortic regurgitation, Mitral regurgitation, Tricuspid regurgitation and Pulmonary regurgitation. It should be noted that the valvular disease may involves calcification of mechanical valves, whether biological or synthetic.

In yet another embodiment, the vascular disorder may be a peripheral venous disease such as Varicose veins and chronic venous insufficiency.

Alternatively, the vascular or valvular degenerative process to be prevented by the method of the invention may be caused by exposure of the cells of the endothelial lining to accumulation of macroscopic amorphous calcium phosphate and hydroxyapatite deposits in the extracellular matrix.

The invention further provides a method for preventing or reducing the risk of developing vascular and valvular degenerative disease which is particularly applicable to patients suffering of Chronic-kidney disease or End stage renal disease.

In another embodiment, the invention provides a method for altering the distribution of NKT cells and CD8 lymphocytes in the aortic valves. More specifically, the invention provides a method for reducing the accumulation of NKT cells and CD8 lymphocytes in the aortic valves of a subject in need thereof and thereby alleviates the calcification in said valves. More specifically, it should be noted that as used herein “reducing the accumulation of NKT cells and CD8 lymphocytes” is meant any reduction of bout 5, 10, 20, 30, 40, 50, 60, 70, 80, 90, 95 or 100% of the cells in the aortic valves, as compared to a suitable control, specifically, control valve without treatment.

Still further the invention provides a method for the preparation of a medicament for the treatment of a vascular or valvular degenerative disorder in a subject in need thereof. The method of the invention may comprise the following steps: (i) providing at least one of: (a) any one of a β-glycolipid, a mixture of at least two β-glycolipids and a substance which increases the intracellular, extracellular or serum level of a naturally occurring β-glycolipid; (b) at least one phosphate inhibitor or any compound that alters the phosphate binding or transporting into or out of any cell or any membrane-or any combination thereof; (c) any combination of (a) and (b) and (ii) admixing any one of these β-glycolipid and/or phosphate inhibitors with at least one of a pharmaceutically acceptable carrier, diluent, excipient and/or additive.

The invention further relates to the use of β-glycolipids, as a supporting medicament for the treatment of a non-vascular calcification-related degenerative disorder.

It should be appreciated that the prevention or reduction of the risk of developing vascular and valvular degenerative disease as well as non-vascular calcification-related degenerative disease, comprises the administration of a prophylactically effective amount of the composition of the invention or of the active ingredients comprised within such composition, to a person at risk of developing a vascular or valvular degenerative disease.

The term “prophylactically effective amount” is intended to mean that amount of a pharmaceutical combined composition that will prevent or reduce the risk of occurrence of the biological or medical event that is sought to be prevented in a tissue, a system, animal or human by a researcher, veterinarian, medical doctor or other clinician.

It should be noted that for the method of treatment and prevention provided in the present invention, said therapeutic effective amount, or dosage, is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the composition of the invention in bodily fluids or tissues. Following successful treatment, it may be desirable to have the patient undergo maintenance therapy to prevent the recurrence of the disease state, wherein the composition of the invention is administered in maintenance doses, once or more daily.

Without being bound by any theory, the inventors hypothesis that β-glycolipids may alleviate vascular and valvular degenerative disorders via alteration of the inflammatory pathway leading to atherosclerosis and via influencing the calcification process. Thus, in non-limiting examples, β-glycolipids may influence the inflammatory process by effecting the expression of certain components of the TNF family signaling pathway, specifically RANK ligand, resulting in inhibition of NFκB activation and the accumulation of CD8 and NK T cells, shown to be involved in mediating valvular injury and in the progression of immune-related diseases [Wu (2007) ibid.; Miyake (2005) ibid. Tupin (2004) ibid. Major (2004) ibid. Zigmond (2007) ibid. Ilan (2007) ibid.]. Moreover, this influence upon RANK ligand expression, also effects matrix calicification. This may be due to among others to an effect upon osteopontin expression however other mechanisms may apply.

Induction of valve calcification by high adenine high-phosphorus diet was associated with a significant increase in RANKL expression in aortic valves. Several cell surface G-proteins coupled receptors are considered to activate calcification process in arterial and bone tissues [Guzman, R. J. J Vasc. Surg. 45 Suppl A: A57-63 (2007)]. The activation of these receptors results in upregulation of RANK/RANK Ligand (RANK/RANKL), which are part of the TNF family pathway, and the NFκB cell machinery [Hofbauer (2001) ibid.; Schoppet (2002) ibid.]. RANK is a transmembrane protein together with its ligand are important in osteoclast differentiation and calcification [Hamdy (2007) ibid.]. The formation of the RANK/RANKL receptor/ligand complex is competitively inhibited by osteoprotegerin (OPG) which is a decoy receptor competing with RANKL [Boyce, B. F., and L. Xing. Arthritis Research & Therapy 9 Suppl 1:S1 (2007)]. OPGL regulates lymph node organogenesis; lymphocyte development and interactions between T cells and dendritic cells in the immune system [Kong, Y. Y., et al. Nature 402:304-309 (1999)]. The OPGL receptor, RANK, is expressed on chondrocytes, osteoclast precursors and mature osteoclasts. OPGL expression in T cells is induced by antigen receptor engagement, which suggests that activated T cells may influence bone metabolism through OPGL and RANK [Kong (1999) ibid.]. Activated T cells can directly trigger osteoclastogenesis through OPGL. RANK provides critical signals necessary for lymph node organogenesis and osteoclast differentiation [Dougall, W. C., et al. Genes & Development 13:2412-2424 (1999); Atkins, G. J., et al. J. Bone Miner Res 21:1339-1349 (2006)].

Recently several studies have shown high expression of RANKL and low expression of OPG in human calcified aortic valves [Shetty (2006) ibid.]. RANKL and OPG are differentially expressed in calcific aortic stenosis [Kaden (2004) ibid.]. In cultured human aortic valve myofibroblasts, RANKL promotes matrix calcification and induces the expression of osteoblast-associated genes, indicating a transition towards an osteogenic phenotype [Kaden (2004) ibid.], supporting a potential role for the RANKL-OPG pathway is regulating AVC. The expression pattern of the RANKL/RANK/OPG system suggests that it may have a regulatory role not only in osteoclastogenesis but also in the calcification of aortic valves [Shetty (2006) ibid. Kaden (2004) ibid.]. OPG was suggested to inhibit calcification by reducing the levels of the RANK/RANKL complex [Dellegrottaglie, S., et al. Current Molecular Medicine 6:515-524 (2006). In addition RANK ligand is a major activator of runx-2, which is the most important transcription factor for osteoblast differentiation [Wu, X. B., et al. The Journal of Clinical Investigation 112:924-934 (2003)]. Administration of IGL led to a significant reduction in RANKL expression. These changes reflect activation of NFκB family members in the valve tissue during calcification as well as prevention of this process by IGL. Suppression of RANKL expression by IGL, inhibited NKT cells accumulation. An inhibitory effect of β-sphingolipids in an NKT-mediated immune damage was described in the Concanavalin A immune mediated hepatitis model [Margalit, M., et al. American Journal of Physiology 289:G917-925 (2005)]. The inventors hypothesize that this inhibition, had a preventive effect on CD8 accumulation in the valve. A similar effect was described in other models [Safadi (2007) ibid. Shimizu, K., et al. The Journal of Experimental Medicine 204(11):2641-2653 (2007)]. The RANK dependent-IGL inhibition of NKT and CD8 lymphocytes was associated with alleviation of the aortic calcification as quantitated by MSCT.

Thus, the invention further provides a method for reducing RANKL expression in a subject in need thereof by administering to said subject an effective amount of at least one of: (a) a β-glycolipid, a mixture of at least two β-glycolipids and a substance which increases the intracellular, extracellular or serum level of a naturally occurring β-glycolipid, or of a composition comprising the same; and (b) at least one phosphate inhibitor or any compound that alters the phosphate binding or transporting into or out of any cell or any membrane or any combination thereof, all resulting in the blockage of the TNF family pathway through reduction of NFκB activation. This may be particularly applicable in the treatment of mammalian subjects suffering of chronic kidney disease or End stage renal disease.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group, of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### Examples

Experimental Procedures

Animals

*Male Sprague-Dawley rats, 8 weeks old, weighing 250-270 g, purchased from Harlan Laboratories Israel, were used as an AVC model.

Animals are maintained in the Animal Core of the Hadassah-Hebrew University Medical School. Rats were administered diet as specified below laboratory and water ad libitum, and kept in 12-hour light/dark cycles. Animal experiments were carried out according to a protocol approved by the Hebrew University Ethics Committee, complying with the Principles of Laboratory and Animal Regulations Established by the National Society of Medical Research.

Preparation of β-glycolipids

β-glucosylceramide (also indicated as GluC or GC) and β-galactosyl-ceramide (also indicated as LacC) were purchased from Avanti Polar Lipids (Alabaster, Ala.), dissolved in ethanol and emulsified in phosphate-buffered saline (PBS). The 1:1 ratio combination of both, IGL, was prepared.

Experimental Group and Study Design

Twenty six rats were studied and divided into three groups. Rats in groups A and B, (n=10 each), were fed exclusively with high-adenine (0.75%), high-phosphate diet (1.5%) (Teklad, Madison, Wis., USA) for 7 weeks, after, which they were fed with normal rat chow for an additional 2 weeks; Rats in control group C, (n=6), were kept on normal laboratory chow. Rats in group B were treated daily IP administration 2.5 mg/kg of IGL. Rats in group A and C, were treated daily with IP administration of 1.5 microliters of PBS. After 9 weeks, all rats were anesthetized, and multislice computed tomography (MSCT) scan were performed for determination of the degree of aortic stenosis. Animals were sacrificed by exsanguinations following blood sample collection from abdominal aorta. Aortic valve tissue was excised, snapped frozen in liquid nitrogen, and kept at −80° C.

Evaluation of the Effect of High Adenine and Phosphorus Diet on Biochemical Profile

A biochemical profile was obtained for all animals in all groups at the fourth and ninth weeks of the study. Serum was analyzed for potassium, phosphate, alkaline phosphate, creatinine, and total cholesterol using VITRO system 5.1 chemistry (Ortho-Clinical Diagnostics, Johnson and Johnson).

Determination of the Effect of Treatment on Degree Apoptosis

Assessment of the effect of β-glycosphingolipids on the inflammatory process was determined by staining of the valves for cleaved capase 3. Formalin-fixed aortic valve tissue at 5 mm cross-sections were used for immunohistochemistry study. Sections were incubated overnight with anti caspase 3 antibody. After phosphate buffer saline wash, sections were incubated with goat anti-rabbit (1:200) secondary antibody_{—} conjugated with Cy5 (Jackson Immunoresearch Laboratories, Inc., West Grove, Pa., USA) for one hour.

Determination of the Effect of Treatment on Degree of Aortic Stenosis by Multislice Computed Tomography

A 64 slice chest. MSCT scanning without contrast was performed on all rats (Brilliance, Philips Medical Systems, Groningen, Netherlands) on the ninth week. Study parameters were: 120 KVp, 300 mAs, slice thickness 0.67 mm, increment 0.3 mm. The scan was analyzed by an operator blinded to the study groups, on an off-line CT workstation. The Agatston Score was calculated by multiplying the area of a calcified lesion with a weighted CT attenuation score dependent on the maximal CT attenuation (HU) within a lesion as previously described [Agatston, A. S., et al. Journal of the American College of Cardiology 15:827-832 (1990)].

Determination of the Effect of Treatment on Aortic Valve Inflammatory Markers:

Isolation of Aortic Valve Lymphocytes:

Aortic valve lymphocytes were isolated as described before with the following modifications. Aortic valve lymphocytes were isolated by crushing the valves through a stainless mesh (size 60, Sigma Chemical Co., St Louis Mo.) [Shibolet, O,. et al. Clinical immunology (Orlando, Fla.) 105:48-56 (2002)]. Cell suspension was placed in a 50 ml tube for 3 minutes and washed twice with cold PBS (1,250 rpm for 10 minutes), and debris was removed. Cells were re-suspended in PBS, cell suspension was placed through a nylon mesh presoaked in PBS, and unbound cells were collected. Cells were washed twice in 45 ml PBS. For Aortic valve isolation, 20 ml of histopague 1077 (Sigma Diagnostics, St Louis, Mo.) was placed underneath the cells suspended in 7 ml of PBS, in a 50-ml tube. The tube was centrifuged in 1,640 rpm for 15 minutes at room temperature. Cells at the interface were collected, diluted in a 50-ml tube, and washed twice with ice-cold PBS (1,250 rpm for 10 minutes).

Flow Cytometry Analysis for Determination of CD4+, CD8+ and NKT Lymphocyte Subsets:

Following lymphocyte isolation, triplicates of 2-5×10⁵ cells/500 μl PBS were placed in Falcon 2052 tubes, incubated with 4 ml of 1% BSA for 10 minutes, and centrifuged at 1400 rpm for 5 minutes. Cells were resuspended in 10 μl FCS with 1:20 FITC-anti mouse CD3 antibody, 1:20 PE-anti mouse CD4 antibody, 1:20 APC-anti mouse CD8 antibody, or 1:20 FITC-anti mouse NK1.1 antibody (NKR-P1C, Pharmingen, USA), and mixed every 10 minutes for 30 minutes. CD4, CD8 and NKT cells were isolated using anti CD3 and anti CD4, anti CD8 and anti NK1.1 respectively. Cells were washed twice in 1% BSA, and kept at 4° C. until reading. For the control group, only 5 μl of 1% BSA was added. Analytical cell sorting was performed on 1×10⁴ cells from each group with a fluorescence-activated cell sorter (FACSTAR plus, Becton Dickinson). Only live cells were counted, and background fluorescence from non-antibody-treated lymphocytes was subtracted. Gates were set on forward- and side-scatters to exclude dead cells and red blood cells. Data were analyzed by the Consort 30 two-color contour plot (Becton Dickinson, Oxnard, Calif.) or CELL Quest programs.

Assessment of the Effect of β-glycolipids on RANK Ligand

Tissue samples were homogenized in ice-cold lysis with PBS. Samples were then sonicated on ice and centrifuged for 10 minutes at 14000 rpm at 4° C. The supernatant was collected and the protein concentration was determined using the Bradford assay. Proteins (40 μg) were separated by SDS-PAGE electrophoresis and subsequently transferred to nitrocellulose membranes. Membranes were blocked with 5% nonfat dry milk in TBS-T and incubated with monoclonal antibody against sRANK (1:250 dillution), (Abcam, Cambridge, UK). Bound antibody was detected with peroxidase-linked dsecondary antibody (Jackson ImmunoResearch, West Grove, Pa.) and a chemiluminescence detection system (Santa Cruz Biotechnology, Calif.). Loading accuracy was evaluated by membrane rehybridization with polyclonal antibody against β-actin (Abcam, Cambridge, UK).

Tissue Analysis

Aortic valve was dissected, fixed in formalin, and embedded in paraffin. Serial cross-sections of the valve were stained using haematoxylin & eosin and with Von-Kossa stains in order to assess the structure and calcium deposits.

Immunohistochemistry Studies

Formalin-fixed aortic valve tissue at 5 mm cross-sections were used for immunohistochemistry studies. The sections were incubated overnight with anti-PIT1 antibody 1:500 (Santa Cruz Biotechnology, Calif.). After phosphate buffer saline wash, the sections were incubated with goat anti-rabbit (1:200) secondary antibody conjugated with Cy5 (Jackson Immunoresearch Laboratories, Inc., West Grove, Pa., USA) for one hour.

Statistical Analysis

Data are presented as mean±the standard error of the mean. Statistical differences between the diet group and control rats were calculated using the student t test. All p values were two-tailed. p value<0.05 was considered significant.

### Example 1

The Effect of β-glycolipids and Combinations thereof on RNAKL Expression

Induction of valve calcification by high adenine high-phosphorus diet was associated with a significant increase in RANKL expression in aortic valves through the activation of G-protein coupled receptors. This activation results in the upregulation of RANK/RANK Ligand which are part of the TNF family pathway, and the NFκB cell machinery. RANK and its ligand are important in osteoclast differentiation and calcification. It should be noted that high expression of RANKL was observed in human calcified aortic valves.

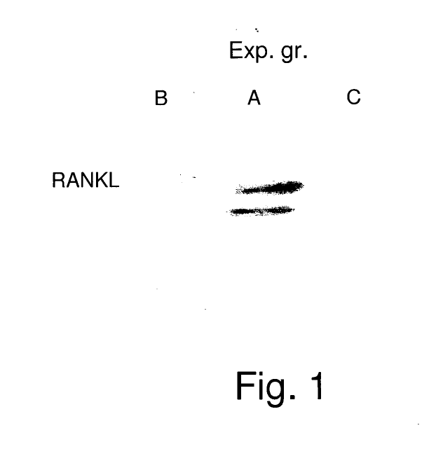

As, shown by the Western blot analysis presented by FIG. 1, induction of valve calcification by high adenine high-phosphorus diet was associated with a significant increase in RANKL expression in valves of rats of group A, as compared to valves from the control group C. As shown by the figure administration of IGL (group B) led to a significant reduction in the, level of RANKL expression.

Without being bound by any theory, these changes may indicate the involvement of NFκB family members in calcification of valve tissue and the prevention of this process by IGL.

### Example 2

The Effect of β-glycolipids and Combinations thereof on Lymphocyte Distribution in Aortic Valve

Aortic valve calcification involves the accumulation of activated T cells and macrophages in aortic valves lesions. Activated CD8+ T cells accumulate closely to endothelial cells in human calcified valves [Wu (2007) ibid.].

NKT cells have been implicated in regulation of adaptive immune responses. Abnormalities in the number and function of NKT cells have been observed in patients with autoimmune diseases. Restricted NKT cells exacerbate atherosclerosis and are proatherogenic [Miyake (2005) ibid; Tupin (2004) ibid. Major (2004) ibid.].

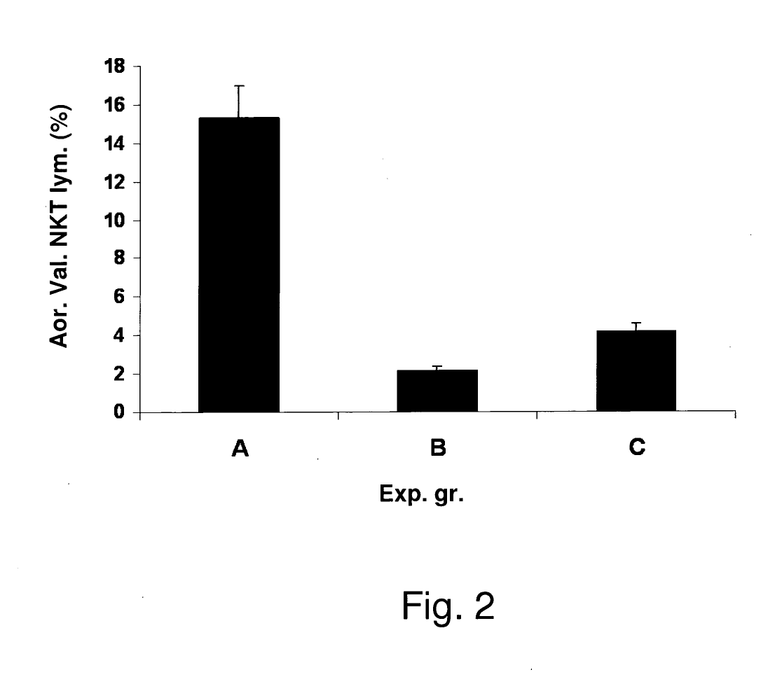

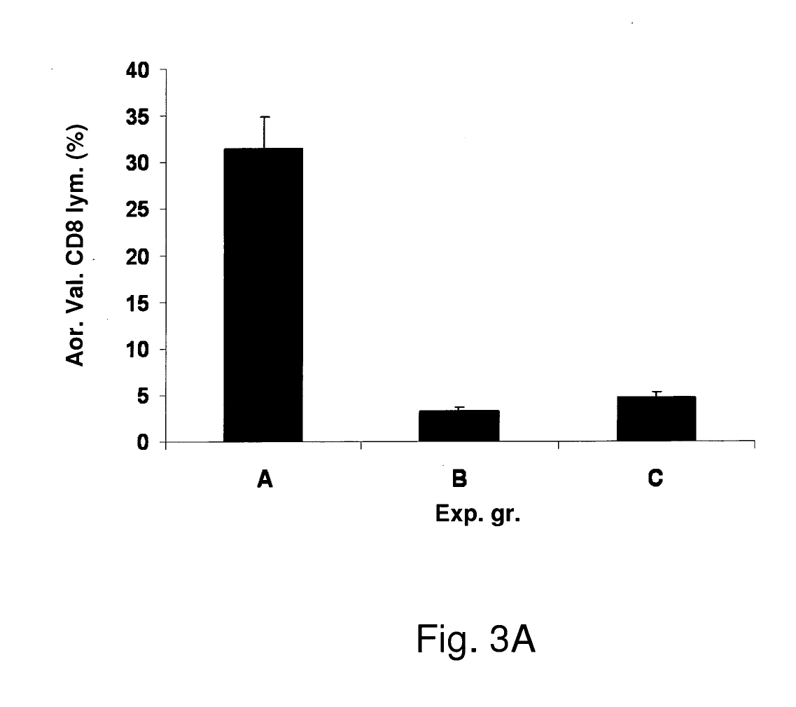

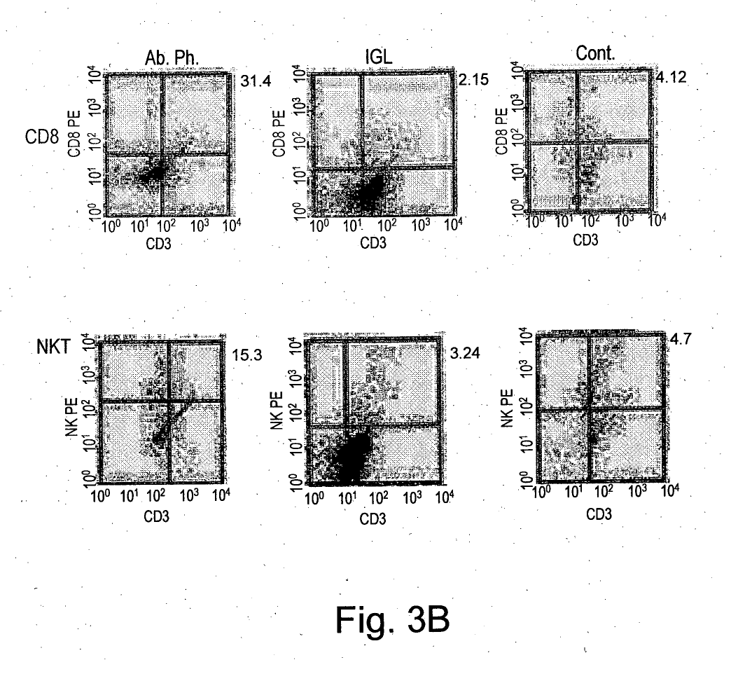

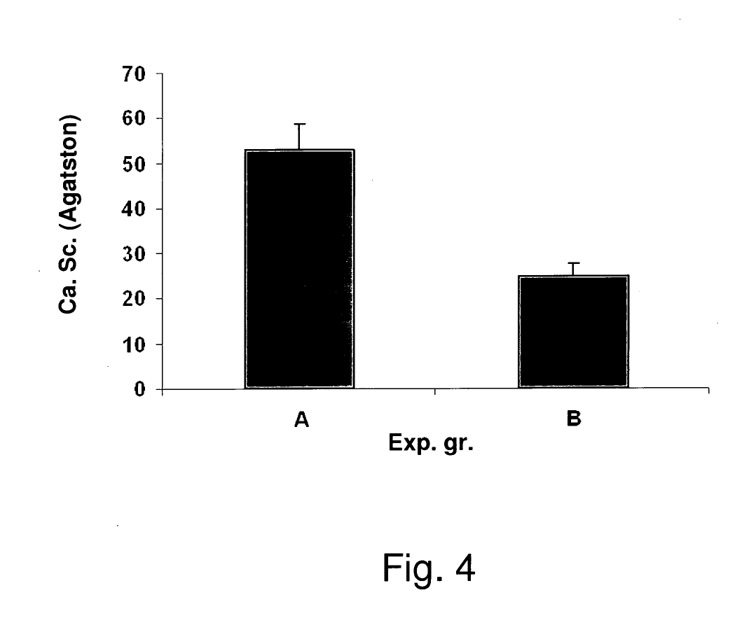

Administration of high adenine high-phosphorus diet was associated with marked changes in CD8 and NKT lymphocyte accumulation in the aortic valve. FACS analysis of aortic valve derived lymphocytes for CD8, NKT (CD3+NK1.1) subsets were performed on all valves of all three experimental groups. As shown by the histogram of FIG. 2, a significant increase in NKT cells was noted in group A valves (15.3%) as compared to 4.7% of the control group C (p<0.005). These results are also presented by the FACS analysis of FIG. 3B. Similarly, as shown by the histogram of FIG. 3A, the high adenine high-phosphorus diet was associated with a significant increase in CD8 lymphocytes (31.4% and 4.12%, for group A and C, respectively, p<0.005).

As clearly shown by both FIGS. 2 and 3, administration of IGL in Group B, significantly altered the lymphocyte distribution, and the lymphocyte distribution pattern was similar to the one noted in rats of control group C. More specifically, as shown in FIG. 2 NKT cells decreased to 2.15%, compared to 15.3% in untreated animals of group A (p<0.005) and in FIG. 3, CD8 cells decreased to 3.24% in the IGL treated group B as compared with 31.4% in group A valves (p<0.005).

### Example 3

β-glycolipids Effect on the Degree of Aortic Stenosis

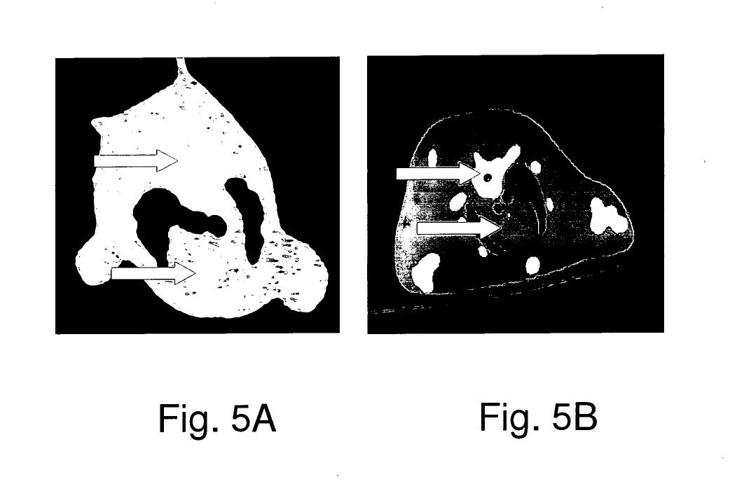

In order to further investigate the mechanism underling the beneficial effect of IGL on biological mechanisms of aortic calcification, the inventors next examined the effect of IGL on aortic stenosis. The IGL induced alteration in RNAKL expression and in CD8 and NKT lymphocyte distribution pattern, were associated with marked alleviation of the aortic stenosis. MSCT was used for assessment of the effect of. IGL treatment on degree of aortic stenosis. Aortic valve calcification was identified in all animals from groups A and B who received a high adenine (0.75%) and high-phosphorus diet (1.5%). No evidence for calcification was noted in rats in control group C. Treatment with IGL exerted a significant decrease in AVC in group B treated animals compared with group A (25 versus 53 Agatston Score, for groups. A and B respectively, p<0.005). FIG. 5 shows a representative MSCT section showing AVC in an animal from group A.

### Example 4

β-glycosphingolipids Ameliorated the Degree of Apoptosis in the Valve

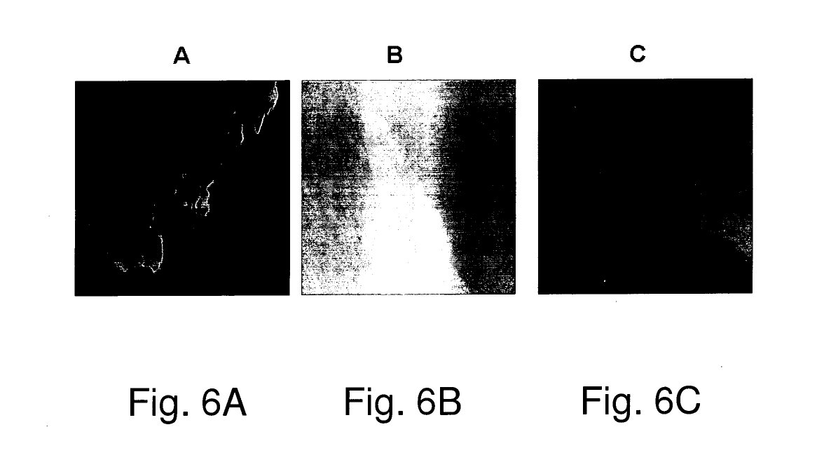

To further investigate the mechanism associated with the effect of β-glycosphingolipids on the AVC process, their involvement in apoptosis has been next examined. Therefore, valves from treated animals were stained for cleaved caspase 3 as a measure of the degree of apoptosis. FIG. 6 shows that treatment with IGL led to significant reduction in the amount of cleaved caspase 3. These data suggest that the beneficial effect may be associated with a decrease in an apoptotic process in the calcified valve.

### Example 5

Effect of High-Adenine, High-Phosphorus Diet on Electrolytes and Kidney Function

A biochemical profile was obtained for all groups; values were taken in groups A and B at 4 weeks (during the adenine diet) and at 9 weeks (2 weeks after cessation of diet), and in the control group at 9 weeks. All rats in groups A and B developed a significant elevation in creatinine and phosphate levels, reflecting renal failure at 4 weeks from the start of the study. There were no significant differences in potassium, alkaline phosphate, or total cholesterol levels. At 9 weeks, 2 weeks after cessation of the adenine diet, both renal failure and hyperphosphatemia resolved.

### Example 6

Decrease in PTH or Phosphate Levels Alleviate the Calcification Process via an Immune Mediated Effect

As firstly shown by the present invention, the renal failure and AVC processes demonstrated by the high phosphate, high adenine model developed by part of the inventors, was shown to be reversible upon treatment with β-glycolipids.

A recent study by part of the inventors demonstrated that the AVC and renal failure processes are dynamic and reversible [Shuvy, M. Cardiovascular Research 79:492-499 (2008)]. More specifically, this study showed that valve calcification resolved after diet cessation in parallel with normalization of renal failure and mineral homeostasis. Moreover, resolution was associated with down-regulation of inflammation and osteoblastic features, as demonstrated by reduction in CD68, osteopontin and osteocalcin expression.

The present inventors have now further explored the calcification process in an attempt to specifically identify targets for interfering and prevention thereof. Therefore, the inventors next examined myofibroblasts obtained from the aortic valve of wild type rats, cultured in 24 well plates.

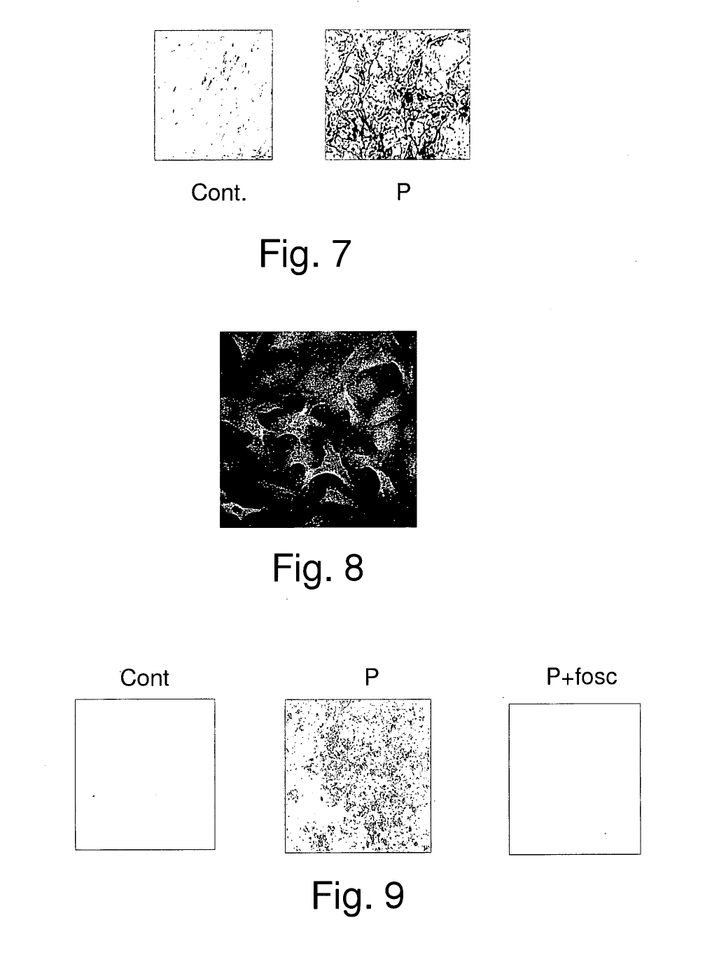

Myofibroblasts were incubated with PTH (1, 10, or 100 μml), or phosphate, 1, 10 or 100 μg/ml) for fourteen days. As shown by the Von kossa (calcium) staining presented by FIG. 7, the phosphate treatment, but not the PTH, induces osteogenic differentiation and calcification of valve myofibroblasts.

In order to explore the exact mechanism of phosphate induced calcification and identify targets for interrupting such process, the inventors next examined the expression of different candidate targets for this process, in valve myofibroblasts. As shown by the immunohistochemical staining of FIG. 8, valve myofibroblasts express the sodium-phosphate co transporter PIT1.

To test the possibility that inhibition of PIT1 would decrease mineralization, valve myofibroblasts were incubated with 10 μg/ml phosphate, or with phosphate and 0.5 mM foscarnet, the specific PIT1 inhibitor for 7 days. As remarkably shown by the Von kossa staining of FIG. 9, cell mineralization has been completely inhibited by the foscarnet treatment.

In order to asses the effect of foscarnet on treating AVC in vivo, the high Adenine, high phosphate rat model was used. Three different groups (10 rats each) were fed daily with the Adenine diet (diet group) for 6 weeks (Group A). Group B (n=9) followed the same protocol and was also treated with 5 mg/kg foscarnet administered intraperitoneally (i.p.). The control group C, received daily chew. Renal function tests, electrolytes were followed for nine weeks. Multislice computed tomography (MSCT) was performed at the ninth week.

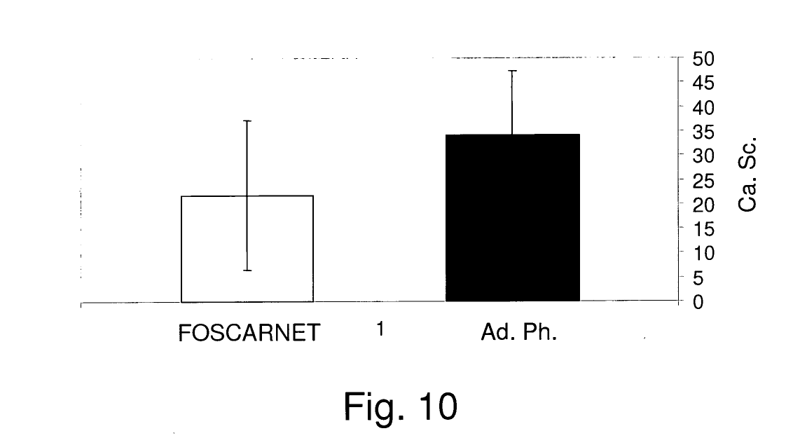

Calcium scores were calculated using the Agatston score. As shown by FIG. 10, all diet group animals (group A) developed aortic valve calcification. No calcium was found in the control group (group C, not shown). Foscarnet treatment led to a significant decrease in AVC (group B) as compared with the diet group A (34.04 versus 21) (P=0.05).

In order to investigate whether the beneficial effect of foscarnet on the calcification process also affects immunomodulatory parameters, the inventors the effect of foscarnet on the levels of cytokines that are known as having a role in osteoblast transformation and maturation. Therefore, levels of TNF-α and IL-6 were next measured in supernatants of myofibroblasts treated with foscarnet. Myofibroblasts treated with foscarnet exhibited' a significant reduction in TNF-α levels, from 69 to 5 pg/ml with no effect on. IL-6 levels. These results clearly indicate the anti-inflammatory effect of foscarnet.

In summary, the present invention demonstrates for the first time the reversibility of the renal failure and AVC processes in response to either modulation of immune response using β-glycolipids or by interfering with the calcification process by reducing phosphate levels using a PIT1 inhibitor, such as the foscarnet. Therefore, addressing both immunomodulatory and calcification aspects in a combined therapy including β-glycolipids and phosphate inhibitors, provides a comprehensive and complete treatment for calcification disorders.

While this invention has been described in terms of some specific examples, many modifications and variations are possible. It is therefore understood that within the scope of the appended claims, the invention may be realized otherwise than as specifically described.

1. A composition comprising a combination of at least one natural or synthetic β-glycolipid, a mixture of at least two β-glycolipids, a substance which increases the intracellular, extracellular or serum level of a naturally occurring β-glycolipid, or any combination or mixture thereof and at least one phosphate inhibitor or any compound that alters the phosphate binding or transporting into or out of any cell or any membrane or any combination thereof, said composition optionally further comprising at least one pharmaceutically acceptable carrier, diluent, excipient and/or additive.

2. The composition according to claim 1, wherein said β-glycolipid is selected from the group consisting of a lactosyl-ceramide, a glucosylceramide, a monosaccharide ceramide, a galactosylceremide, a gal-gal-glucosyl-ceramide, GM2 ganglioside, GM3 ganglioside, globoside or any other β-glycolipid, any natural or synthetic analogs derivatives thereof and any combinations or mixtures thereof and wherein a mixture of said β-glycolipids comprises at least two β-glycolipids at a quantitative ratio between about 1:1 to 1:1000.

3. (canceled)

4. The composition according to claim 2, wherein said mixture comprises β-lactosyl-ceramide and β-glucosyl-ceramide at a quantitative ratio between about any one of 1:1 to 1:1000 and 1:1 to 1000:1.

5. The composition according to claim 1, wherein said phosphate inhibitor or any compound that alters the phosphate binding or transporting into or out of any cell or any membrane or any combination thereof is any one of a sodium-phosphate co transporter PIT 1 inhibitor, a phosphate inhibitor and a Parathyroid hormone (PTH) inhibitor.

6. The composition according to claim 5, wherein said PIT 1 inhibitor is foscarnet.

7. The composition according to claim 1, wherein said composition is for the treatment of calcification related degenerative disorder.

8. A kit for achieving a therapeutic effect in a subject in need thereof comprising:
a. at least one natural or synthetic β-glycolipid, mixture of at least two β-glycolipids, a substance which increases the intracellular, extracellular or serum level of a naturally occurring β-glycolipid; or any combination or mixture thereof, or a pharmaceutically acceptable derivative thereof and a pharmaceutically acceptable carrier or diluent optionally, in a first unit dosage form;
b. at least one phosphate inhibitor or any compound that alters the phosphate binding or transporting into or out of any cell or any membrane or any combination thereof and a pharmaceutically acceptable carrier or diluent, optionally, in a second unit dosage form; and
c. container means for containing said first and second dosage forms.

9. The kit according to claim 8, wherein said subject is suffering from a calcification related degenerative disorder selected from a vascular or valvular degenerative disorder and calcification related disorders in visceral organs.

10. A method for the treatment or prevention of a calcification related degenerative disorder in a subject in need thereof, which method comprises the step of administering to said subject a therapeutically effective amount of at least one of:
a. at least one of a natural or synthetic β-glycolipid, a mixture of at least two β-glycolipids, a substance which increases the intracellular, extracellular or serum level of a naturally occurring β-glycolipid;
b. at least one phosphate inhibitor or any compound that alters the phosphate binding or transporting into or out of any cell or any membrane or any combination thereof; and
c. a composition or kit comprising (a) or (b) or of any combinations thereof.

11. The method according to claim 10, wherein said calcification related degenerative disorder is any one of a vascular or valvular degenerative disorder and calcification related disorders in visceral organs.

12. The method according to claim 11, wherein said β-glycolipid is selected from the group consisting of a lactosyl-ceramide, a glucosylceramide, a monosaccharide ceramide, galactosylceremide, a gal-gal-glucosyl-ceramide, GM2 ganglioside, GM3 ganglioside, globoside or any other β-glycolipid, any natural or synthetic analogs derivatives thereof and any combinations or mixtures thereof and wherein a mixture of said β-glycolipids comprises at least two J3-glycolipids at a quantitative ratio between about 1:1 to 1:1000.

13. (canceled)

14. The method according to claim 12, wherein said mixture comprises β-lactosyl-ceramide and β-glucosyl-ceramide at a quantitative ratio between about any one of 1:1 to 1:1000 and 1:1 to 1000:1.

15. The method according to claim 10, wherein said phosphate inhibitor or any compound that alters the phosphate binding or transporting into or out of any cell or any membrane or any combination thereof, is any one of a sodium-phosphate co transporter PIT 1 inhibitor, a phosphate inhibitor and a Parathyroid hormone (PTH) inhibitor.

16. The method according to claim 15, wherein said PIT 1 inhibitor is foscarnet.

17. The method according to claim 10, wherein the method comprises the step of administering to said subject a therapeutically effective amount of at least one of a natural or synthetic β-glycolipid, a mixture of at least two β-glycolipids, a substance which increases the intracellular, extracellular or serum level of a naturally occurring β-glycolipid or any combination and mixtures thereof or of a composition comprising the same.

18. The method according to claim 10, wherein the method comprises the step of administering to said subject a therapeutically effective amount of at least one phosphate inhibitor or any compound that alters the phosphate binding or transporting into or out of any cell or any membrane or any combination and mixtures thereof or of a composition comprising the same.

19. The method according to claim 10, wherein at least one of the β-glycolipid or any mixture or combination thereof and the phosphate inhibitor or any compound that alters the phosphate binding or transporting into or out of any cell or any membrane or any combination thereof is administered alone, or in combination with other active ingredient/s, whether administered in combination, serially or simultaneously and wherein said administering step comprises oral, intravenous, intramuscular, subcutaneous, intraperitoneal, parenteral, transdermal, intravaginal, intranasal, mucosal, sublingual, topical, rectal or subcutaneous administration, or any combination thereof.

20.-22. (canceled)

23. A method for protection of cells of the endothelial lining from a vascular or valvular degenerative process comprising the step of contacting said cells with a protective effective amount of at least one of (a) any one of β-glycolipid, a mixture of at least two β-glycolipids and a substance which increases the intracellular, extracellular or serum level of a naturally occurring β-glycolipid; (b) at least one phosphate inhibitor or any compound that alters the phosphate binding or transporting into or out of any cell or any membrane or any combination thereof; (c) a composition or a kit comprising the same or any combinations thereof.

24. A method for decreasing RANKL expression in a subject in need thereof, and thereby blocking TNF family pathway, wherein said reduction of RANKL expression reduces NFκB activation, said method comprising the step of administering to said subject an effective amount of at least one of: (a) a β-glycolipid, a mixture of at least two β-glycolipids and a substance which increases the intracellular, extracellular or serum level of a naturally occurring β-glycolipid; (b) at least one phosphate inhibitor or any compound that alters the phosphate binding or transporting into or out of any cell or any membrane or any combination thereof; and (c) a composition comprising the same.

25. The method according to claim 24, wherein said subject is a mammalian subject suffering of chronic kidney disease or End stage renal disease.
